(19) United States
(12) Patent Application Publication
(10) Pub. No.: US 2025/0027124 A1
(43) Pub. Date: Jan. 23, 2025

(54) MICROORGANISM AND METHOD FOR THE IMPROVED PRODUCTION OF VALINE

(71) Applicant: METABOLIC EXPLORER, SAINT BEAUZIRE (FR)

(72) Inventors: Perrine VASSEUR, SAINT-LAURE (FR); Thomas DESFOUGERES, DISSAY (FR); Céline RAYNAUD, SAINT BEAUZIRE (FR)

(73) Assignee: METABOLIC EXPLORER, SAINT BEAUZIRE (FR)

(21) Appl. No.: 18/711,309

(22) PCT Filed: Nov. 17, 2022
(86) PCT No.: PCT/EP2022/082274

§ 371 Date: May 17, 2024

(30) Foreign Application Priority Data
Nov. 17, 2021 (EP) 21306600.4

(51) Int. Cl. C12P 13/08; C07K 14/245; C12N 9/88

(52) U.S. Cl. CPC C12P 13/08; C07K 14/245; C12N 9/88; C12Y 403/01019

(57) ABSTRACT

The present invention relates to a microorganism genetically modified for improved production of valine, wherein the microorganism overexpresses a ilvA gene coding a threonine deaminase and/or exhibits an increased threonine deaminase activity and comprises a mutated argP gene coding DNA-binding transcriptional dual regulator. The present invention also relates to a method for the production of valine using said microorganism.

### FIELD OF INVENTION

The present invention relates to a microorganism genetically modified for the improved production of valine and to a method for the improved production of valine using said microorganism.

### BACKGROUND OF THE INVENTION

Amino acids are used in many industrial fields, including the food, animal feed, cosmetics, pharmaceutical, and chemical industries and have an annual worldwide market growth rate of an estimated 5 to 7% (Leuchtenberger, et al., 2005).

Branched chain amino acids also function as precursors in the synthesis of herbicides and antibiotics, such as polyketides. Unlike most other amino acids which are metabolized in the liver, the branched-chain amino acids are metabolized mainly in muscles, such that they are used as energy sources for moving the body.

Branched chain amino acids (BCAA) may be produced via chemical synthesis, extraction from protein hydrolysates, or microbial fermentation. Of these techniques, fermentation is the most commonly used today, due to the associated economic and environmental advantages. In particular, fermentation provides a useful way of using abundant, renewable, and/or inexpensive materials as the main source of carbon. Furthermore, while both D- and L-enantiomers are generated in equimolar amounts when using chemical synthesis, requiring additional downstream isolation of the L-enantiomer, fermentation produces only the L-enantiomer.

Among these, the valine branched chain amino acid is particularly important for the nutrition of humans and a number of livestock species as being among the nine essential amino acids that cannot be synthesized in mammals. L-valine has been usually manufactured by bacterial fermentation, employing mutant strains of Corynebacterium glutamicum and Escherichia coli. So far, most BCAA production strains have been developed by random mutagenesis.

These microbial strains have a shortcoming in that it is difficult to additionally improve the strains, because it is difficult to understand the precise physiological metabolism thereof. Thus, in the art to which the present invention pertains, there is a need to develop microorganisms having high abilities for valine productivity, thus providing novel methods for the production of valine at a reduced cost.

### BRIEF DESCRIPTION OF THE INVENTION

The present invention concerns a microorganism genetically modified for the production of valine and methods for the production of valine using said microorganism. The microorganism genetically modified for the production of valine notably overexpresses a ilvA gene coding a threonine deaminase and/or exhibits an increased threonine deaminase activity as compared to the expression level and/or threonine deaminase activity in a corresponding wild-type microorganism. Also, this microorganism comprises a mutated argP gene coding DNA-binding transcriptional dual regulator.

Indeed, the inventors have surprisingly found that such a microorganism shows improved production of valine, by overexpressing the ilvA gene and/or increasing threonine deaminase activity by contrast with the prior art methods in which the ilvA gene expression or corresponding enzymatic activity is either not modified or rather attenuated and even deleted.

Preferably, in the genetically modified microorganism according to the invention the ilvA gene is overexpressed in the recombinant microorganism such as by modifying the promoter regulating the expression of the ilvA gene, by increasing the number of copies of the ilvA gene present in the microorganism, or by overexpressing the ilvA gene from a plasmid, by improving stability of the ilvA mRNA or increasing IlvA protein quantity by optimization of Ribosome Binding Site, preferably by mutating the promoter regulating the expression of the ilvA gene.

More preferably, the ilvA gene is overexpressed in the recombinant microorganism by increasing the number of copies of the ilvA gene present in the microorganism, leading to two copies of the gene.

Preferably, the microorganism further comprises further overexpresses a fepA gene coding ferric enterobactin outer membrane transporter and/or exhibits an increased ferric enterobactin outer membrane transporter activity as compared to the expression level and/or ferric enterobactin outer membrane transporter activity in a corresponding wild-type microorganism.

Preferably, the microorganism further comprises a deletion of at least one gene selected from the group consisting of IdhA, adhE and mgsA.

Preferably, the microorganism further comprises an overexpression of at least one gene selected from the group consisting of vdh, ilvD, ilvC, ilvB, and ilvN*

Preferably, the microorganism belongs to the family of bacteria Enterobacteriaceae, Corynebacteriaceae, Bacillaceae, Streptococcae or Lactobacillus, or to the family of fungus such as Hemiascomycetus, filamentous fungus or yeast.

Preferably, said Enterobacteriaceae bacterium is Escherichia coli, said Corynebacteriaceae bacterium is Corynebacterium glutamicum or said Bacillaceae is Bacillus subtilis said Streptococcae is Streptococcus thermophiles, said Lactobacillus is Lactobacillus lactis, said Hemiascomycetus yeast is Saccharomyces cerevisiae or Yarrowia lipolytica and said filamentous fungus is Tricchoderma rezeii or Aspergillus niger, more preferably wherein said microorganism is Escherichia coli.

Preferably, the microorganism is the one with the number CNCM I-5911, deposited on Oct. 19, 2022 at the Collection Nationale de Cultures de Microorganismes, Pasteur Institute, 25 Rue du Docteur Roux, 75724 PARIS Cedex 15, FRANCE.

The present invention further comprises a method for the production of valine comprising the steps of:
a) culturing a microorganism genetically modified for the production of valine described in any of the embodiments provided herein in an appropriate culture medium comprising a source of carbon, and
b) recovering valine from the culture medium.

Preferably, the recovering of valine comprises at least the steps of:
a) clarification of the fermentation medium for removing the insoluble organic impurities,
b) treatment of the product of the preceding step on an adsorbent such as activated charcoal for removing soluble organic impurities and inorganic impurities,
c) evaporation of water and crystallization of the product obtained,
d) recovering valine.

Preferably, the source of carbon is selected from arabinose, fructose, galactose, glucose, lactose, maltose, sucrose, xylose, or any polysaccharide such as starch, cellulose or hemicellulose, and any combination thereof.

### DETAILED DESCRIPTION

Before describing the present invention in detail, it is to be understood that the invention is not limited to particularly exemplified microorganisms and/or methods and may, of course, vary. It is also to be understood that the terminology used herein is for the purpose of describing particular embodiments of the invention only, and is not intended to be limiting. The invention will be limited only by the appended claims.

All publications, patents, and patent applications cited herein, whether supra or infra, are hereby incorporated by reference in their entirety. Furthermore, the practice of the present invention employs, unless otherwise indicated, conventional microbiological and molecular biological techniques that are within the skill of the art. Such techniques are well-known to the skilled person, and are fully explained in the literature.

Unless defined otherwise, all technical and scientific terms used herein have the same meanings as commonly understood by one of ordinary skill in the art to which this invention belongs. Although any materials and methods similar or equivalent to those described herein can be used to practice or test the present invention, preferred materials and methods are provided.

It must be noted that as used herein and in the appended claims, the singular forms “a,” “an,” and “the,” include the plural reference unless the context clearly dictates otherwise. Thus, for example, a reference to “a microorganism” includes a plurality of such microorganisms, and a reference to “an endogenous gene” is a reference to one or more endogenous genes, and so forth.

The terms “comprise,” “comprises,” and “comprising” are used in an inclusive sense, i.e. to specify the presence of the stated features but not to preclude the presence or addition of further features in various embodiments of the invention.

A first aspect of the invention relates to a microorganism genetically modified for the production of valine.

The term “microorganism,” as used herein, refers to a living microscopic organism, which may be a single cell or a multicellular organism and which can generally be found in nature. In the present context, the microorganism is preferably a bacterium, yeast, or fungus. Preferably, the microorganism of the invention is selected from the Enterobacteriaceae, Corynebacteriaceae, Bacillaceae, Streptococcae or Lactobacillus, or to the family of fungus such as Hemiascomycetus, filamentous fungus or yeast. More preferably, the microorganism of the invention is a species of Escherichia, Corynebacterium, Bacillus, Streptococcus, Lactobacillus. Even more preferably, said Enterobacteriaceae bacterium is Escherichia coli, said Corynebacteriaceae bacterium is Corynebacterium glutamicum or said Bacillaceae is Bacillus subtilis said Streptococcae is Streptococcus thermophiles, said Lactobacillus is Lactobacillus lactis, said Hemiascomycetus yeast is Saccharomyces cerevisiae or Yarrowia lipolytica and said filamentous fungus is Tricchoderma rezeii or Aspergillus niger. Most preferably, the microorganism of the invention is Escherichia coli.

The terms “recombinant microorganism” or “microorganism genetically modified” are used interchangeably herein and refer to a microorganism or a strain of microorganism that has been genetically modified or genetically engineered. This means, according to the usual meaning of these terms, that the microorganism of the invention is not found in nature and is genetically modified when compared to the “parental” microorganism from which it is derived. The “parental” microorganism may occur in nature (i.e. a wild-type microorganism) or may have been previously modified. The recombinant microorganism of the invention may notably be modified by the introduction, deletion and/or modification of genetic elements. Such modifications can be performed, for example, by genetic engineering, by adaptation, wherein a microorganism is cultured in conditions that apply a specific stress on the microorganism and induce mutagenesis, and/or by forcing the development and evolution of metabolic pathways by combining directed mutagenesis and evolution under specific selection pressure.

A microorganism may notably be modified to modulate the expression level of an endogenous gene or the activity of the corresponding enzyme or transcription factor. The term “endogenous gene” means that the gene was present in the microorganism before any genetic modification. Endogenous genes may be overexpressed by introducing heterologous sequences in addition to, or to replace, endogenous regulatory elements. Endogenous gene expression levels, protein expression levels, or the activity of the encoded protein, can also be increased or attenuated by introducing mutations into the coding sequence of a gene or into non-coding sequences. These mutations may be synonymous, when no modification in the corresponding amino acid occurs, or non-synonymous, when the corresponding amino acid is altered. Synonymous mutations do not have any impact on the function of translated proteins, but may have an impact on the regulation of the corresponding genes or even of other genes, if the mutated sequence is located in a binding site for a regulator factor. Non-synonymous mutations may have an impact on the function or activity of the translated protein as well as on regulation depending on the nature of the mutated sequence.

In particular, mutations in non-coding sequences may be located upstream of the coding sequence (i.e. in the promoter region, in an enhancer, silencer, or insulator region, in a specific transcription factor binding site) or downstream of the coding sequence. Mutations introduced in the promoter region may be in the core promoter, proximal promoter, or distal promoter. Mutations may be introduced by site-directed mutagenesis using, e.g., Polymerase Chain Reaction (PCR), by random mutagenesis techniques e.g. via mutagenic agents (Ultra-Violet rays or chemical agents like nitrosoguanidine (NTG) or ethylmethanesulfonate (EMS)), DNA shuffling, error-prone PCR, or using culture conditions that apply a specific stress on the microorganism and induce mutagenesis. The insertion of one or more supplementary nucleotide(s) in the region located upstream of a gene can notably modulate gene expression.

A particular way of modulating endogenous gene expression is to exchange the endogenous promoter of a gene (e.g., wild-type promoter) with a stronger or weaker promoter to upregulate or downregulate expression of the endogenous gene. The promoter may be endogenous (i.e. originating from the same species) or exogenous (i.e. originating from a different species). It is well within the ability of the person skilled in the art to select an appropriate promoter for modulating the expression of an endogenous gene. Such a promoter may be, for example, a Ptrc, Ptac, or Plac promoter, or the PR or PL lambda promoters. The promoters may be “inducible” by a particular compound or by specific external conditions, such as temperature or light.

A particular way of modulating endogenous protein activity is to introduce nonsynonymous mutations in the coding sequence of the corresponding gene, e.g. according to any of the methods described above. A non-synonymous amino acid mutation that is present in a transcription factor may notably alter binding affinity of the transcription factor toward a cis-element, alter ligand binding to the transcription factor, etc.

A microorganism may also be genetically modified to express one or more exogenous (i.e. heterologous) genes so as to express or overexpress the corresponding gene product (e.g. an enzyme). An “exogenous” or “heterologous” gene as used herein refers to a gene encoding a protein or polypeptide that is introduced into a microorganism in which said gene does not naturally occur. A “heterologous gene” as used herein also refers to a gene that was endogenous to a microorganism (i.e. present in the microorganism prior to any genetic modification) but that, when introduced into the microorganism, is not introduced at the location where the endogenous gene is/was located. More particularly, the heterologous gene may be an endogenous gene in cases where expression of endogenous gene itself in the microorganism is reduced as compared to the microorganism in which the gene naturally occurs (e.g. due to a mutation, a complete or partial deletion of the gene, a modification in the transcriptional regulation of the gene, etc.). In particular, the endogenous gene may no longer be expressed or may be expressed at very low levels. The exogenous gene may be directly integrated into the chromosome of the microorganism, or be expressed extra-chromosomally within the microorganism by plasmids or vectors. For successful expression, exogenous gene(s) must be introduced into the microorganism with all of the regulatory elements necessary for their expression or be introduced into a microorganism that already comprises all of the regulatory elements necessary for their expression. The genetic modification or transformation of microorganisms with one or more exogenous genes is a routine task for those skilled in the art.

One or more copies of a given exogenous gene can be introduced on a chromosome by methods well-known in the art, such as by genetic recombination. When a gene is expressed extra-chromosomally, it can be carried by a plasmid or a vector. Different types of plasmids are notably available, which may differ in respect to their origin of replication and/or their copy number in the cell. For example, a microorganism transformed by a plasmid can contain 1 to 5 copies of the plasmid, about 20 copies, or even up to 500 copies, depending on the nature of the selected plasmid. A variety of plasmids having different origins of replication and/or copy numbers are well-known in the art and can be easily selected by the skilled person for such purposes, including, e.g., pTrc, pACYC184, pBR322, pUC18, pUC19, pKC30, pRep4, pHS1, pHS2, or pPLc236.

It should be understood that, in the context of the present invention, when an exogenous gene encoding a protein of interest is expressed in a microorganism, a synthetic version of this gene may preferably be constructed by replacing non-preferred codons or less preferred codons with preferred codons of said microorganism which encode the same amino acid. Indeed, it is well-known in the art that codon usage varies between microorganism species, and that this may impact the recombinant expression level of the protein of interest. To overcome this issue, codon optimization methods have been developed, and are extensively described by Graf et al. (2000), Deml et al. (2001) and Davis & Olsen (2011). Several software programs have notably been developed for codon optimization determination such as the GeneOptimizer® software (Lifetechnologies) or the OptimumGene™ software of (GenScript). In other words, the exogenous gene encoding a protein of interest is preferably codon-optimized for expression in the microorganism.

The terms “expressing,” “overexpressing,” or “overexpression” of a protein of interest, such as an enzyme, refer herein to an increase in the expression level and/or activity of said protein in a microorganism, as compared to the corresponding parent microorganism that does not comprise the modification(s) present in the genetically modified microorganism, also named corresponding wild-type microorganism. In some cases, the level of expression may be similar to that of the parent microorganism. In other cases, the level of expression may be superior to that of the parent microorganism. In cases where a parent microorganism does not comprise the protein of interest, the term “expression” or “overexpression” refers to the presence of the protein of interest, as compared to its absence in the parent microorganism.

In contrast, the terms “attenuating” or “attenuation” of a protein of interest refer to a decrease in the expression level and/or activity of said protein in a microorganism, as compared to the parent microorganism. The attenuation of expression can notably be due to either the exchange of the wild-type promoter for a weaker natural or synthetic promoter or the use of an agent reducing gene expression, such as antisense RNA or interfering RNA (RNAi), and more particularly small interfering RNAs (siRNAs) or short hairpin RNAs (shRNAs). Promoter exchange may notably be achieved by the technique of homologous recombination (Datsenko & Wanner, 2000). The complete attenuation of the expression level and/or activity of a protein of interest means that expression and/or activity is abolished, thus, the expression level of said protein is null. The complete attenuation of the expression level and/or activity of a protein of interest may be due to the complete suppression of the expression of a gene. This suppression can be either an inhibition of the expression of the gene, a deletion of all or part of the promoter region necessary for expression of the gene, or a deletion of all or part of the coding region of the gene. A deleted gene can notably be replaced by a selection marker gene that facilitates the identification, isolation, and purification of the modified microorganism. As a non-limiting example, suppression of gene expression may be achieved by the technique of homologous recombination, which is well-known to the person skilled in the art (Datsenko & Wanner, 2000).

Modulating the expression level of one or more proteins may thus occur by altering the expression of one or more endogenous genes that encode said protein within the microorganism as described above or by introducing one or more heterologous genes that encode said protein into the microorganism.

The term “expression level” as used herein, refers to the amount (e.g. relative amount, concentration) of a protein of interest (or of the gene encoding said protein) expressed in a microorganism, which is measurable by methods well-known in the art. The level of gene expression can be measured by various known methods including Northern blotting, quantitative RT-PCR, and the like. Alternatively, the level of expression of the protein coded by said gene may be measured, for example by SDS-PAGE, HPLC, LC/MS, and other quantitative proteomic techniques (Bantscheff et al., 2007), or, when antibodies against said protein are available, by Western Blot-Immunoblot (Burnette, 1981), Enzyme-linked immunosorbent assay (e.g. ELISA) (Engvall and Perlman, 1971), protein immunoprecipitation, immunoelectrophoresis, and the like. The copy number of an expressed gene can be quantified, for example, by restricting chromosomal DNA followed by Southern blotting using a probe based on the gene sequence, fluorescence in situ hybridization (FISH), RT-qPCR, and the like.

Overexpression of a given gene or the corresponding protein may be verified by comparing the expression level of said gene or protein in the genetically modified organism to the expression level of the same gene or protein in a control microorganism that does not have the genetic modification (i.e. the parental microorganism).

The terms “activity” or “function” as used herein in the context of an enzyme designate the reaction that is catalyzed by said enzyme for converting its corresponding substrate(s) into another molecule(s) (i.e. product(s)). As is well-known in the art, the activity of an enzyme may be assessed by measuring its catalytic efficiency and/or Michaelis constant. Such an assessment is described for example in Segel, 1993, in particular on pages 44-54 and 100-112, incorporated herein by reference.

The microorganism genetically modified for improved production of valine provided herein overexpresses a ilvA gene coding a threonine deaminase and/or exhibits an increased threonine deaminase activity as compared to the expression level and/or threonine deaminase activity in a corresponding wild-type microorganism. Indeed, the inventors have surprisingly shown that the above genetic modifications improve valine production notably in productivity and yield, as compared to a parent microorganism that does not comprise these modifications. Improved valine production in this microorganism is particularly surprising as ilvA gene expression or threonine deaminase activity is generally either not modified or attenuated and even deleted, viz. null in prior art methods with improvement of valine production.

In the microorganism genetically modified for the production of valine provided herein, any well-known prior art methods may be used for overexpressing the ilvA gene expression or increasing the threonine deaminase activity. “Overexpression” or “overexpressing” is also used to designate increasing transcription of a gene in the microorganisms. Increasing transcription of a gene can be achieved by increasing the number of copies of the gene and/or using a promoter leading to a higher level of expression of the gene.

For example, the ilvA gene may be overexpressed by modifying the promoter regulating the expression of the ilvA gene, by increasing the number of copies of the ilvA gene present in the microorganism, or by overexpressing the ilvA gene from a plasmid, by improving stability of the ilvA mRNA or increasing IlvA protein quantity by optimization of Ribosome Binding Site.

Preferably, the ilvA gene expression is overexpressed, and in particular by increasing the number of copies of the ilvA gene present in the microorganism.

For increasing the number of copies of the gene in the microorganism, the gene is encoded chromosomally or extrachromosomally. When the gene is located on the chromosome, several copies of the gene can be introduced on the chromosome by methods of recombination, known by the expert in the field (including gene replacement). When the gene is located extra-chromosomally, it may be carried by different types of plasmids that differ with respect to their origin of replication and thus their copy number in the cell, as described above.

In one preferred embodiment, the number of copies of the ilvA gene present in the microorganism genetically modified according to the invention is of at least two copies of the gene. Two, three, four or five copies of the gene are particularly preferred. Up to 10 or 15 copies may also be considered. More preferably, from 2 to 5 copies of the ilvA gene are present in the microorganism genetically modified of the invention. Most preferably, the ilvA gene is overexpressed in the microorganism genetically modified so as to lead to two copies of the gene.

Increasing translation of the mRNA can be achieved by modifying the Ribosome Binding Site (RBS). A RBS is a sequence on mRNA that is bound by the ribosome when initiating protein translation. It can be either the 5′ cap of a mRNA in eukaryotes, a region 6-7 nucleotides upstream of the start codon AUG in prokaryotes (called the Shine-Dalgarno sequence), or an internal ribosome entry site (IRES) in viruses. By modifying this sequence, it is possible to change the protein translation initiation rate, proportionally alter its production rate, and control its activity inside the cell. The same RBS sequence will not have the same impact according to the nature of the mRNA. It is possible to optimize the strength of an RBS sequence to achieve a targeted translation initiation rate by using the software RBS CALCULATOR (Salis, 2011).

Improving stability of the mRNA can be achieved by decreasing mRNA turnover can be achieved by modifying the gene sequence of the 5′-untranslated region (5′-UTR) and/or the coding region, and/or the 3′-UTR (Carrier and Keasling, 1999).

On a preferred embodiment, the microorganism overexpresses an ilvA gene coding a threonine deaminase of SEQ ID NO: 2, or a functional fragment or functional variant thereof.

The term “functional fragment” of a protein of reference having a biological activity of interest (e.g. of an enzyme having threonine deaminase activity), as used herein refers to parts of the amino acid sequence of an enzyme, said parts comprising at least all the regions essential for exhibiting the biological activity of said protein. These parts of sequences can be of various lengths, provided that the biological activity of the amino acid sequence of reference is retained by said parts. In other words, the functional fragments of the enzymes provided herein are enzymatically active.

“Functional variants” of an enzyme described herein (e.g. of an enzyme having threonine deaminase activity) include, but are not limited to, enzymes having amino acid sequences which are at least 60% identical after alignment to the amino acid sequence encoding the corresponding reference enzyme. According to the present invention, the variant preferably has at least 70%, 80%, 85%, 90%, 95%, 96%, 97%, 98%, 99%, or 100% amino acid sequence identity to the protein described herein (e.g. an IlvA protein). Thus, the enzyme having threonine deaminase activity preferably has at least 70%, 80%, 85%, 90%, 95%, 96%, 97%, 98%, 99%, or 100% sequence identity to the amino acid sequence of SEQ ID NO: 2. More preferably, the gene encoding the enzyme having threonine deaminase activity has at least 70%, 80%, 85%, 90%, 95%, 96%, 97%, 98%, 99%, or 100% sequence identity to the nucleotide sequence of SEQ ID NO: 1. As a non-limiting example, means of determining sequence identity are further provided below.

Increasing an enzymatic activity can also be obtained by improving the protein catalytic efficiency or decreasing protein turnover or decreasing messenger RNA (mRNA) turnover or increasing transcription of the gene or increasing translation of the mRNA.

Improving protein catalytic efficiency means increasing the kcat and/or decreasing the Km for a given substrate and/or a given cofactor, and/or increasing the Ki for a given inhibitor. kcat, Km and Ki are Michaelis-Menten constants that the person skilled in the art is able to determine (Segel, 1993). Decreasing protein turnover means stabilizing the protein. Methods to improve protein catalytic efficiency and/or decrease protein turnover are well-known by the person skilled in the art. Those include rational engineering with sequence and/or structural analysis and directed mutagenesis, as well as random mutagenesis and screening. Mutations can be introduced by site-directed mutagenesis by usual methods like Polymerase Chain Reaction (PCR), or by random mutagenesis techniques, such as use of mutagenic agents (Ultra-Violet rays or chemical agents like nitrosoguanidine (NTG) or ethylmethanesulfonate (EMS)) or use of PCR techniques (DNA shuffling or error-prone PCR). Stabilizing the protein can also be achieved by adding a peptide sequence called “tag” either at the N-terminus or the C-terminus of the protein. Tags are well known from the person skilled in the art. For instance, a Glutathione-S-Transferase (GST) can be used to stabilize a protein.

Genes and proteins are identified herein using the denominations of the corresponding genes in E. coli (e.g. E. coli K12 MG1655 having the Genbank accession number U00096.3) unless otherwise specified. However, in some cases use of these denominations has a more general meaning according to the invention and covers all of the corresponding genes and proteins in microorganisms. This is notably the case for the genes and proteins described herein that are not endogenous to the microorganism of the invention (i.e. that are heterologous), such as IlvA. As a particular example, and as indicated above, functional variants of IlvA, are comprised herein, as are mutants and functional fragments thereof. Particular aspects are further detailed below.

PFAM (protein family database of alignments and hidden Markov models; http://www.sanger.ac.uk/Software/Pfam/) represents a large collection of protein sequence alignments. Each PFAM makes it possible to visualize multiple alignments, see protein domains, evaluate distribution among organisms, gain access to other databases, and visualize known protein structures.

COGs (clusters of orthologous groups of proteins; http://www.ncbi.nlm.nih.gov/COG/) are obtained by comparing protein sequences from 43 fully sequenced genomes representing 30 major phylogenic lines. Each COG is defined from at least three lines, which permits the identification of former conserved domains.

The means of identifying similar sequences and their percent identities are well-known to those skilled in the art, and include in particular the BLAST programs, which can be used from the website http://www.ncbi.nlm.nih.gov/BLAST/with the default parameters indicated on that website. The sequences obtained can then be exploited (e.g., aligned) using, for example, the programs CLUSTALW (http://www.ebi.ac.uk/clustalw/) or MULTALIN (http://prodes.toulouse.inra.fr/multalin/cgi-bin/multalin.pl), with the default parameters indicated on those websites.

Using the references given on GenBank for known genes, the person skilled in the art is able to determine the equivalent genes in other organisms, bacterial strains, yeasts, fungi, mammals, plants, etc. This routine work is advantageously done using consensus sequences that can be determined by carrying out sequence alignments with genes derived from other microorganisms, and designing degenerate probes to clone the corresponding gene in another organism. These routine methods of molecular biology are well-known to those skilled in the art.

Sequence identity between amino acid sequences can be determined by comparing a position in each of the sequences which may be aligned for the purposes of comparison. When a position in the compared sequences is occupied by the same amino acid, then the sequences are identical at that position. A degree of sequence identity between proteins is a function of the number of identical amino acid residues at positions shared by the sequences of said proteins.

As a non-limiting example, to determine the percentage of identity between two amino acid sequences, the sequences are aligned for optimal comparison. For example, gaps can be introduced in the sequence of a first amino acid sequence for optimal alignment with the second amino acid sequence. The amino acid residues at corresponding amino acid positions are then compared. When a position in the first sequence is occupied by the same amino acid residue as the corresponding position in the second sequence, the molecules are identical at that position.

The percentage of identity between the two sequences is a function of the number of identical positions shared by the sequences. Hence % identity=number of identical positions/total number of overlapping positions×100.

Optimal alignment of sequences may be conducted by the global alignment algorithm of Needleman and Wunsch (1972), by computerized implementations of this algorithm (such as CLUSTAL W) or by visual inspection. The best alignment (i.e., resulting in the highest percentage of identity between the compared sequences) generated by the various methods is selected.

In other words, the percentage of sequence identity is calculated by comparing two optimally aligned sequences, determining the number of positions at which the identical amino acid occurs in both sequences to yield the number of matched positions, dividing the number of matched positions by the total number of positions and multiplying the result by 100 to yield the percentage of sequence identity.

The above definitions and preferred embodiments related to the functional fragments and functional variants of proteins apply mutatis mutandis to nucleotide sequences, such as genes, encoding a protein of interest (i.e. an enzyme having threonine deaminase activity).

In addition to the modification described above, the genetically modified microorganism of the invention may comprise one or more additional modifications among those described below. Said modifications are advantageous as they may notably further improve valine production, titer, and/or yield. One or more of said modifications may notably promote valine synthesis, inhibit the use of valine as a substrate in downstream metabolic pathways, promote stable accumulation of valine, or inhibit toxic accumulation of valine in the microorganism.

According to the present invention, the microorganism further comprises a mutated argP gene coding DNA-binding transcriptional dual regulator.

Preferably, the argP gene itself is mutated. More preferably, the argP gene is modified so as to lead a substitution of the amino acid at position 128 or a corresponding position. The most preferred embodiment on this point is a substitution of Glu which is at position 128 by Asp (otherwise referred to herein as an “argP* mutant”) or a corresponding position. The position of the amino acid residue indicated correspond to that provided in SEQ ID NO: 4. Also, the argP gene encodes a transcriptional regulator which leads to various modulations (from overexpression to decreased expression) on target genes expression by binding directly to their promoter or control region. Among the target genes, the following may be mentioned: gdhA, dapB, dapD, lysP, lysA, lysC, asd, dnaAN-recF, nrdAB-yfaE, argP, argO, argK . . . . Therefore, all the modifications on target genes expression occurring due to the substitution of the amino acid at position 128 of argP or a corresponding position, compared to the usual modulations where argP is not mutated, viz. wild-type, should be considered as having the same improvement on valine production and yield than the substitution of this same amino acid position on argP itself.

Corresponding positions can notably be determined by those skilled in the art using manual alignment or by using an alignment program (e.g., BLASTP). Corresponding positions can also be based on structural alignments, for example by using computer-simulated alignments of protein structures. The fact that an amino acid of a polypeptide corresponds to an amino acid in the disclosed sequence means that when the polypeptide and the disclosed sequence are aligned, a standard alignment calculation method such as a GAP calculation method is used. A corresponding amino acid may notably be identified when conserved amino acids are aligned such that the sequences have maximized identity or homology. As used herein, “in a corresponding position” refers to a position of interest in a nucleic acid molecule or protein (i.e. nucleotide base or amino acid residue number) relative to a position in a reference nucleic acid molecule or protein. Positions of interest relative to positions in reference proteins can be, for example, allelic variants, heterologous proteins, amino acid sequences of the same protein in other species, etc. Corresponding positions can be determined by comparing and aligning sequences such that the number of paired nucleotides or amino acid residues is maximized. For example, identity between sequences may be greater than 95%, 96%, 97%, 98%, or more particularly greater than 99%. The position of interest is then given the number assigned in the sequence of the reference nucleic acid molecule or polypeptide.

Thus according to the present invention, the microorganism overexpresses a ilvA gene coding a threonine deaminase and/or exhibits an increased threonine deaminase activity as compared to the expression level and/or threonine deaminase activity in a corresponding wild-type microorganism, combined to a mutated argP gene coding DNA-binding transcriptional dual regulator as described above.

According to another preferred embodiment, the microorganism further overexpresses a fepA gene coding ferric enterobactin outer membrane transporter and/or exhibits an increased ferric enterobactin outer membrane transporter activity as compared to the expression level and/or ferric enterobactin outer membrane transporter activity in a corresponding wild-type microorganism.

As mentioned herein, any method known in the art may be used to overexpress the fepA gene in the microorganism according to the invention. Thus, any technical means which leads to an increased fepA gene expression or an increased ferric enterobactin outer membrane transporter activity as compared to the expression level and/or ferric enterobactin outer membrane transporter activity in a corresponding wild-type microorganism, may be used to genetically modify the microorganism in the context of the present invention. In particular and only as examples, by genetic modification, such as by modifying the promoter regulating the expression of the fepA gene, by increasing the number of copies of the fepA gene present in the microorganism, or by overexpressing the fepA gene from a plasmid, by improving stability of the fepA mRNA or increasing FepA protein quantity by optimization of Ribosome Binding Site, preferably by mutating the promoter regulating the expression of the fepA gene. In fact, any modification able to lead to an increased fepA gene expression or an increased ferric enterobactin outer membrane transporter activity, as compared to the expression level and/or ferric enterobactin outer membrane transporter activity in a corresponding wild-type microorganism, is included in the scope of the present invention.

Preferably, the microorganism further comprises an increased expression of the fepA gene by at least one base replacement in the promoter sequence regulating the expression of the fepA gene. More preferably, the at least one base replacement, and in particular one base replacement, is carried out just downstream of the “−10 box” (also known as “Pribnow box”) which is located at about 10 pairs of nucleotides upstream the transcription start point and is constituted of 6 nucleotides optionally with some variations but generally being of sequence TATAAT. In particular, one nucleotide replacement is carried out 3 nucleotides upstream from the transcription start of the promoter controlling the expression of the fepA gene as set forth in SEQ ID NO: 7. More preferably, thymine nucleotide at position-3 from the transcription start of the fepA promoter is replaced by cytosine nucleotide as set forth in SEQ ID NO: 8.

In another advantageous embodiment of the invention, the microorganism overexpresses a ilvA gene coding a threonine deaminase and/or exhibits an increased threonine deaminase activity as compared to the expression level and/or threonine deaminase activity in a corresponding wild-type microorganism, combined to an overexpression of a fepA gene coding ferric enterobactin outer membrane transporter and/or an increased ferric enterobactin outer membrane transporter activity as described above.

In a still advantageous embodiment of the invention, the microorganism overexpresses a ilvA gene coding a threonine deaminase and/or exhibits an increased threonine deaminase activity as compared to the expression level and/or threonine deaminase activity in a corresponding wild-type microorganism, combined to a mutated argP gene coding DNA-binding transcriptional dual regulator as described above, and combined to an overexpression of a fepA gene coding ferric enterobactin outer membrane transporter and/or an increased ferric enterobactin outer membrane transporter activity as described above.

According to still another preferred embodiment, the microorganism further comprises an attenuation of the expression of one or more of the following proteins: lactate dehydrogenase (LdhA), alcohol dehydrogenase (AdhE), methylglyoxal synthase (MgsA), fumarate reductase enzyme complex (FrdABCD), pyruvate formate lyase (PflAB), acetate kinase (AckA) and phosphate acetyltransferase (Pta) and/or branched chain amino acid transporters (BrnQ and LivKHMGF). Said genes are notably endogenous in E. coli.

Preferably, LdhA has at least 80%, 90%, 95%, or 100% sequence similarity or sequence identity with the sequence of SEQ ID NO: 10. Preferably, AdhE has at least 80%, 90%, 95%, or 100% sequence similarity or sequence identity with the sequence of SEQ ID NO: 12. Preferably, MgsA has at least 80%, 90%, 95%, or 100% sequence similarity or sequence identity with the sequence of SEQ ID NO: 14. Preferably, FrdA, FrdB, FrdC, and FrdD have at least 80%, 90%, 95%, or 100% sequence similarity or sequence identity with the sequence of SEQ ID NO: 16, 18, 20 and 22, respectively. Preferably, PfIA and PfIB have at least 80%, 90%, 95%, or 100% sequence similarity or sequence identity with the sequence of SEQ ID NO: 24 and 26, respectively. Preferably, AckA has at least 80%, 90%, 95%, or 100% sequence similarity or sequence identity with the sequence of SEQ ID NO: 28. Preferably Pta has at least 80%, 90%, 95%, or 100% sequence similarity or sequence identity with the sequence of SEQ ID NO: 30. Preferably BrnQ has at least 80%, 90%, 95%, or 100% sequence similarity or sequence identity with the sequence of SEQ ID NO: 32. Preferably LivK, LivH, LivM, LivG and LivF have at least 80%, 90%, 95%, or 100% sequence similarity or sequence identity with the sequences of SEQ ID NO: 34, 36, 38, 40 and 42, respectively.

Preferably, attenuation of expression results from a partial or complete deletion of the gene encoding said protein (i.e., IdhA, adhE, mgsA, frdABCD, pflAB, ackA-pta, brnQ and/or livKHMGF genes). Preferably, the genetically modified microorganism of the invention further comprises a deletion of at least one gene selected from the group consisting of IdhA, adhE and mgsA.

Preferably, the IdhA gene has at least 80%, 90%, 95%, or 100% sequence identity with the sequence of SEQ ID NO: 9. Preferably, the adhE gene has at least 80%, 90%, 95%, or 100% sequence identity with the sequence of SEQ ID NO: 11. Preferably, the mgsA gene has at least 80%, 90%, 95%, or 100% sequence identity with the sequence of SEQ ID NO: 13. Preferably, the frdABCD genes have at least 80%, 90%, 95%, or 100% sequence identity with the sequences of SEQ ID NOs: 15, 17, 19, and 21, respectively. Preferably, the pflAB genes have at least 80%, 90%, 95%, or 100% sequence identity with the sequences of SEQ ID NOs: 23 and 25, respectively. Preferably, the ackA-pta genes have at least 80%, 90%, 95%, or 100% sequence identity with the sequences of SEQ ID NOs: 27 and 29, respectively. Preferably, the brnQ gene has at least 80%, 90%, 95%, or 100% sequence identity with the sequence of SEQ ID NO: 31. Preferably, the livKHMGF genes have at least 80%, 90%, 95%, or 100% sequence identity with the sequences of SEQ ID NOs: 33, 35, 37, 39 and 41, respectively.

The microorganism for the production of valine may further comprise an overexpression of one or more of the following proteins: ketol-acid reductoisomerase (NADP(+)) (IlvC), dihydroxy-acid dehydratase (IlvD), acetolactate synthase (IlvBN*), valine dehydrogenase (Vdh), branched-chain-amino-acid aminotransferase (IlvE) and L-valine exporter (YgaZH). Preferably, the dehydrogenase is a leucine or valine dehydrogenase.

Preferably, IlvC has at least 80%, 90%, 95%, or 100% sequence similarity or sequence identity with the sequence of SEQ ID NO: 44. Preferably, IlvD has at least 80%, 90%, 95%, or 100% sequence similarity or sequence identity with the sequence of SEQ ID NO: 46. Preferably, IlvB and IlvN* have at least 80%, 90%, 95%, or 100% sequence similarity or sequence identity with the sequence of SEQ ID NOs: 48 and 52, respectively, with IlvN* comprising the substitutions G20D, V21D and M22F in cases where the sequence is not 100% identical to SEQ ID NO: 52. Preferably, Vdh has at least 80%, 90%, 95%, or 100% sequence similarity or sequence identity with the sequence of SEQ ID NO: 54. Preferably, IlvE has at least 80%, 90%, 95%, or 100% sequence similarity or sequence identity with the sequence of SEQ ID NO: 57. Preferably, YgaZ and YgaH have at least 80%, 90%, 95%, or 100% sequence similarity or sequence identity with the sequence of SEQ ID NOs: 59 and 61, respectively.

Preferably, the overexpression of said one or more proteins results from an overexpression of the gene coding said protein (i.e., ilvC, ilvD and/or ilvBN* genes). Preferably, the genetically modified microorganism of the invention further comprises an overexpression of at least one gene selected from the group consisting of vdh, ilvD, ilvC, ilvB, and ilvN*

Preferably, the ilvC gene has at least 80%, 90%, 95%, or 100% sequence identity with the sequence of SEQ ID NO: 43. Preferably, the ilvD gene has at least 80%, 90%, 95%, or 100% sequence identity with the sequence of SEQ ID NO: 45. Preferably, the ilvB and ilvN* genes have at least 80%, 90%, 95%, or 100% sequence identity with the sequences of SEQ ID NOs: 47 and 51, respectively, wherein the ilvN* gene codes for an amino acid having the substitutions G20D, V21D and M22F with reference to the wild-type protein having the sequence SEQ ID NO: 50. Preferably, vdh has at least 80%, 90%, 95%, or 100% sequence identity with the sequence of SEQ ID NO: 53. Preferably, ilvE has at least 80%, 90%, 95%, or 100% sequence identity with the sequence of SEQ ID NO: 56. Preferably, ygaZ and ygaH have at least 80%, 90%, 95%, or 100% sequence identity with the sequence of SEQ ID NOs: 58 and 60, respectively.

As mentioned above, the microorganism of the invention may belong to the family of bacteria, fungus or yeast.

Preferably, said microorganism belongs to the family of bacteria Enterobacteriaceae, Corynebacteriaceae, Bacillaceae, Streptococcae or Lactobacillus, or to the family of fungus such as Hemiascomycetus, filamentous fungus or yeast.

According to preferred embodiments, said Enterobacteriaceae bacterium is Escherichia coli, said Corynebacteriaceae bacterium is Corynebacterium glutamicum or said Bacillaceae is Bacillus subtilis said Streptococcae is Streptococcus thermophiles, said Lactobacillus is Lactobacillus lactis, said Hemiascomycetus yeast is Saccharomyces cerevisiae or Yarrowia lipolytica and said filamentous fungus is Tricchoderma rezeii or Aspergillus niger, and more preferably said microorganism is Escherichia coli.

Also, the microorganism according to the present invention may be genetically modified so as to comprise either a modified endogenous gene/enzyme or heterologous gene/enzyme. Preferably, the microorganism comprises endogenous gene or enzyme having threonine deaminase activity, more preferably comprises endogenous ilvA gene encoding threonine deaminase.

In a further aspect, when the microorganism as described herein is unable to use sucrose as a carbon source, said microorganism is modified to be able to use sucrose as a carbon source. Preferably, proteins involved in the import and metabolism of sucrose are overexpressed. Preferably, the following proteins are overexpressed:
CscB sucrose permease (SEQ ID NO: 63), CscA sucrose hydrolase (SEQ ID NO: 65), CscK fructokinase (SEQ ID NO: 67), and CscR csc-specific repressor (SEQ ID NO: 69), or
ScrA Enzyme II of the phosphoenolpyruvate-dependent phosphotransferase system (SEQ ID NO: 75), ScrK gene encodes ATP-dependent fructokinase (SEQ ID NO: 71), ScrB sucrose 6-phosphate hydrolase (invertase) (SEQ ID NO: 77), ScrY sucrose porine (SEQ ID NO: 73), and ScrR sucrose operon repressor (SEQ ID NO: 79).

Preferably, genes coding for said proteins are overexpressed according to one of the methods provided herein. Preferably, the E. coli microorganism overexpresses:
the heterologous cscBKAR genes of E. coli EC3132 (cscB gene SEQ ID NO: 62; cscK gene SEQ ID NO: 66; cscA gene SEQ ID NO: 64 and cscR gene SEQ ID NO: 68), or
the heterologous scrKYABR genes of Salmonella sp (scrK gene SEQ ID NO: 70; scrY gene SEQ ID NO: 72; scrA gene SEQ ID NO: 74; scrB gene SEQ ID NO: 76 and scrR gene SEQ ID NO: 78).

In a preferred aspect, the microorganism according to the present invention is the one with the number CNCM I-5911, deposited on Oct. 19, 2022 at the Collection Nationale de Cultures de Microorganismes, Pasteur Institute, 25 Rue du Docteur Roux, 75724 PARIS Cedex 15, FRANCE.

A second object of the invention relates to a method for the production of valine using the microorganism described herein. Said method comprises the steps of:
a) culturing a microorganism genetically modified for the production of valine as described herein in an appropriate culture medium comprising a source of carbon, and
b) recovering valine from the culture medium.

More specifically, the invention relates to a method for the improved fermentative production of valine using the microorganism described herein. According to the invention, the terms “fermentative process,” “fermentative production,” “fermentation,” or “culture” are used interchangeably to denote the growth of microorganism. This growth is generally conducted in fermenters with an appropriate growth medium adapted to the microorganism being used.

An “appropriate culture medium” designates a medium (e.g., a sterile, liquid media) comprising nutrients essential or beneficial to the maintenance and/or growth of the cell such as carbon sources or carbon substrates, nitrogen sources, for example, peptone, yeast extracts, meat extracts, malt extracts, urea, ammonium sulfate, ammonium chloride, ammonium nitrate, and ammonium phosphate; phosphorus sources, for example, monopotassium phosphate or dipotassium phosphate; trace elements (e.g., metal salts), for example magnesium salts, cobalt salts, and/or manganese salts; as well as growth factors such as amino acids and vitamins. In particular, the inorganic culture medium for E. coli can be of identical or similar composition to an M9 medium (Anderson, 1946), an M63 medium (Miller, 1992), or a medium such as defined by Schaefer et al. (1999).

The term “source of carbon,” “carbon source,” or “carbon substrate” according to the present invention refers to any carbon source capable of being metabolized by a microorganism wherein the substrate contains at least one carbon atom. According to the present invention, said source of carbon is preferably at least one carbohydrate, and in some cases a mixture of at least two carbohydrates. CO₂ is not a carbohydrate because it does not contain hydrogen.

The term “carbohydrate” refers to any carbon source capable of being metabolized by a microorganism and containing at least one carbon atom, two atoms of hydrogen and one atom of oxygen. The one or more carbohydrates may be selected from among the group consisting of: monosaccharides such as glucose, fructose, mannose, xylose, arabinose, galactose, and the like, disaccharides such as sucrose, cellobiose, maltose, lactose, and the like, oligosaccharides such as raffinose, stacchyose, maltodextrins, and the like, polysaccharides such as cellulose, hemicellulose, starch, and the like, methanol, formaldehyde, and glycerol. Preferred carbon sources are arabinose, fructose, galactose, glucose, lactose, maltose, sucrose, xylose, or any polysaccharide such as starch, cellulose or hemicellulose, or any combination thereof, more preferably glucose.

The term “recovering” as used herein designates the process of separating or isolating the produced valine by using conventional laboratory techniques known to the person skilled in the art. Recovering valine according to step b) of the method described herein may comprise a step of filtration, desalination, cation exchange, liquid extraction, crystallization, or distillation, or combinations thereof. Valine may be recovered from both culture medium and microorganisms, or from only one or the other. Preferably, valine is recovered from at least the culture medium. The volume of culture medium may be reduced for example via ceramic membrane filtration. Valine may furthermore be recovered either during culturing of the microorganism by in situ product recovery including extractive fermentation, or after fermentation is finished. Microorganisms may notably be removed by passing through a device, preferably through a filter with a cut-off in the range from 5 to 200 kDa, where solid/liquid separation takes place. It is also feasible to employ a centrifuge, a suitable sedimentation device, or a combination of these devices, it being especially preferred to first separate at least part of the microorganisms by sedimentation and subsequently to feed the fermentation broth, from which the microorganisms have been at least partially removed, to ultrafiltration or to a centrifugation device. After the microorganisms have been removed, valine present in the remaining culture medium may be recovered. Valine may be recovered from microorganisms separately. Recovery of valine from microorganism may notably involve lysis or disruption by heating to induce valine release from microorganisms.

In this second object of the invention, the microorganism which is used according to the method for the production of valine is the one with the number CNCM I-5911, deposited on Oct. 19, 2022 at the Collection Nationale de Cultures de Microorganismes, Pasteur Institute, 25 Rue du Docteur Roux, 75724 PARIS Cedex 15, FRANCE.

More preferably, the recovering of valine according to step b) comprises at least the step of:
a) clarification of the fermentation medium for removing the insoluble organic impurities,
b) treatment of the product of the preceding step on an adsorbent such as activated charcoal for removing soluble organic impurities and inorganic impurities,
c) evaporation of water and crystallization of the product obtained,
d) recovering valine.

Those skilled in the art are able to define the culture conditions for the microorganisms according to the invention. In particular the bacteria are fermented at a temperature between 20° C. and 55° C., preferably between 25° C. and 40° C., more preferably between about 30° C. to 37° C., even more preferably about 37° C.

This process can be carried out either in a batch process, in a fed-batch process, or in a continuous process. It can be carried out under aerobic, micro-aerobic, or anaerobic conditions, or a combination thereof (for example, aerobic conditions followed by anaerobic conditions).

“Under aerobic conditions” means that oxygen is provided to the culture by dissolving the gas into the liquid phase. This could be obtained by (1) sparging oxygen containing gas (e.g. air) into the liquid phase or (2) shaking the vessel containing the culture medium in order to transfer the oxygen contained in the head space into the liquid phase. The main advantage of the fermentation under aerobic conditions is that the presence of oxygen as an electron acceptor improves the capacity of the strain to produce more energy under the form of ATP for cellular processes. Therefore, the strain has its general metabolism improved.

Micro-aerobic conditions are defined as culture conditions wherein low percentages of oxygen (e.g. using a mixture of gas containing between 0.1 and 10% oxygen, completed to 100% with nitrogen), is dissolved into the liquid phase.

Anaerobic conditions are defined as culture conditions wherein no oxygen is provided to the culture medium. Strictly anaerobic conditions are obtained by sparging an inert gas like nitrogen into the culture medium to remove traces of other gas. Nitrate can be used as an electron acceptor to improve ATP production by the strain and improve its metabolism.

The production of valine by the microorganism in the culture broth can be determined unambiguously by standard analytical means known by those skilled in the art. As a non-limiting example, valine may be quantified using isocratic HPLC (Pleissner et al., 2011) or nuclear magnetic resonance.

### EXAMPLES

The present invention is further defined in the following examples. It should be understood that these examples, while indicating preferred embodiments of the invention, are given by way of illustration only. The person skilled in the art will readily understand that these examples are not limitative and that various modifications, substitutions, omissions, and changes may be made without departing from the scope of the invention.

### Methods

The protocols used in the following examples are:
Protocol 1 (Chromosomal modifications by homologous recombination, selection of recombinants and antibiotic cassette excision flanked by FRT sequences) and protocol 2 (Transduction of phage P1) used in this invention have been fully described in patent application WO2013/001055 (see in particular the “Examples Protocols” section and Examples 1 to 8, incorporated herein by reference).
Protocol 3: Construction of recombinant plasmids.

Recombinant DNA technology is well described and known to the person skilled in the art. Briefly, DNA fragments were PCR amplified using oligonucleotides (that the person skilled in the art will be able to define) and E. coli MG1655 genomic DNA or an adequate synthetically synthesized fragment was used as a matrix. The DNA fragments and chosen plasmid were digested with compatible restriction enzymes (that the person skilled in the art is able to define), then ligated and transformed into competent cells. Transformants were analyzed and recombinant plasmids of interest were verified by DNA sequencing.
Protocol 4: Evaluation of L-valine fermentation performance

Production strains were evaluated in bioreactor using both media MM_VAB10 and MM_VAB20 (Table 1) for valine production, adjusted to pH 6.8. The MM_VAB10 medium is dedicated to monitor the strain ability to produce valine at initial stage. The MM_VBA20 medium is used to demonstrate the impact of genetic optimizations at high valine content. A 50 mL preculture was grown at 30° C. for 16 hours in a rich medium (LB medium with 5 g·L⁻¹ glucose). It was used to inoculate a 200 mL culture to an OD₆₀₀ of 0.5. When necessary, antibiotics were added to the medium (spectinomycin and chloramphenicol at a final concentration of 50 mg. L⁻¹ and 30 mg·L⁻¹, respectively). The temperature of the cultures was 39° C. The cultures were stopped when the glucose was totally consumed within a maximum culture duration of 50 hours. Extracellular amino acids were quantified by HPLC after OPA/Fmoc derivatization and other relevant metabolites were analyzed using HPLC with refractometric detection (organic acids).

TABLE 1
Composition of MM_VAB10 and MM_VAB20 media

| | MM_VAB10 | MM_VAB20 |
|---|---|---|
| Compound | Concentration (g · L⁻¹) | Concentration (g · L⁻¹) |
| Citric acid•H₂O | 1.00 | 1.00 |
| MgSO₄•7H₂O | 1.00 | 1.00 |
| CaCl₂•2H₂O | 0.04 | 0.04 |
| CoCl₂•6H₂O | 0.0080 | 0.0080 |
| MnSO₄•H₂O | 0.0200 | 0.0200 |
| CuCl₂•2H₂O | 0.0020 | 0.0020 |
| H₃BO₃ | 0.0010 | 0.0010 |
| Na₂MoO₄•2H₂O | 0.0004 | 0.0004 |
| ZnSO₄•7H₂O | 0.0040 | 0.0040 |
| Na₂HPO₄ | 2.00 | 2.00 |
| K₂HPO₄ | 2.00 | 2.00 |
| (NH₄)₂HPO₄ | 8.00 | 8.00 |
| (NH₄)₂SO₄ | 5.00 | 5.00 |
| NH₄Cl | 0.13 | 0.13 |
| FeSO₄•7H₂O | 0.01 | 0.01 |
| Thiamin | 0.0116 | 0.0116 |
| MOPS | 40 | 40 |
| Glucose | 30 | 30 |
| Valine | 0 | 20 |

In these cultures, the valine yield (YVal) was expressed as follows:

$$YVal ( \frac{g}{g} ) = \frac{Valine produced ( g )}{Glucose consummed ( g )} \times 100$$

and the valine productivity (PVal) was expressed as follows:

$$PVal ( \frac{g}{L \times h} ) = \frac{Valine produced ( g )}{Culture volume ( L ) \times Culture duration ( h )}$$

### Example 1: Valine Producing Strains Carrying More than 1 Copy of ilvA Gene have Improved Valine Production Performance

Construction of Valine Producing Strains with Variable Copy Number of ilvA Gene

### Construction of Strain 1

According to protocols 1, 2 and 3, the E. coli MG1655 strain 1 was obtained by sequentially:
knocking out the lactate dehydrogenase (IdhA gene, SEQ ID NO: 9), the alcohol dehydrogenase (adhE gene, SEQ ID NO: 11) and the methylglyoxal synthase (mgsA gene, SEQ ID NO: 13),
replacing the acetohydroxy acid synthase I small regulatory subunit (ilvN gene, SEQ ID NO: 49) by valine feedback resistant (FBR) protein (substitutions G20D, V21D and M22F for IlvN*FBR protein (SEQ ID NO: 52)—Park et al., 2012), and overexpressed it by replacing native promoter by an artificial Ptrc promoter (Brosius et al, 1985),
overexpressing on pCL1920 vector (Lerner & Inouye, 1990) the following genes organized in operon under the control of the PR promoter together with the cI857 allele of the thermosensitive repressor of lambda phage (SEQ ID NO: 80) (amplified from the pFC1 vector, Mermet-Bouvier & Chauvat, 1994):
   the vdh gene (SEQ ID NO: 53) coding for the valine dehydrogenase of Streptomyces aureofaciens (SEQ ID NO: 54, Uniprot A0A1E7N318); more precisely, the vdh gene was synthetically synthesized with the codon usage being optimized to E. coli (SEQ ID NO: 55).
   the ilvD gene (SEQ ID NO: 45) coding for IlvD dihydroxy-acid dehydratase (SEQ ID NO: 46),
   the ilvC gene (SEQ ID NO: 43) coding for IlvC ketol-acid reductoisomerase (SEQ ID NO: 44),
   the ilvBN genes coding for both subunits of the acetohydroxy acid synthase I, with ilvN* FBR allele (ilvB gene SEQ ID NO: 47; ilvN* gene SEQ ID NO: 51), giving rise to plasmid 1.

This strain possesses one copy of ilvA gene (SEQ ID NO: 1) coding for threonine dehydratase (SEQ ID NO: 2), the endogenous one.

### Construction of Strain 2

To construct the strain 2, an additional copy of the ilvA gene with its promoter was integrated in a pseudogene of strain 1, choose preferentially among those cited in Application US2012/0252077 according to protocols 1 and 2.

### Construction of Strain 3

To construct the strain 3, the ilvA gene and its promoter were cloned into pACYC plasmid (Bartolomé et al, 1991) giving rise to plasmid 2 which was introduced in strain 1.

Improvement of Valine Production Related to ilvA Gene Copies into Strain 1 Background

Strains 1 to 3 were grown according to protocol 4. Valine productivity and yield were measured.

TABLE 2
Valine productivity and yield of strains with
one, two or more than 2 copies of ilvA gene.

| | Culture medium | | | | | |
|---|---|---|---|---|---|---|
| | MM_VAB10 | | | MM_VAB20 | | |
| | Strain | | | | | |
| | 1 | 2 | 3 | 1 | 2 | 3 |
| | ilvA copy number | | | | | |
| | 1 | 2 | >2 | 1 | 2 | >2 |
| Productivity | Reference 1 | ++ | ≈ | Reference 3 | +++ | +++ |
| (g/L/h) | | | | | | |
| Yield | Reference 2 | ≈ | ≈ | Reference 4 | ++ | ++ |
| (g/g) | | | | | | |

In MM_VAB10 medium condition, the valine productivity and the valine yield of strain 1, carrying 1 copy of ilvA gene, are referred as « reference 1 » and « reference 2 », respectively, and in MM_VAB20 medium condition, the valine productivity and the valine yield of strain 1, carrying 1 copy of ilvA gene, are referred as « reference 3 » and « reference 4 », respectively. The symbol « ≈ » indicates an increase lesser than 10%, the symbol « ++ » indicates an increase between 30 and 100%, and the symbol « +++ » indicates an increase greater than 100%, compared to appropriate reference.

In MM_VAB10 medium condition, the valine productivity and the valine yield of strain 1, carrying 1 copy of ilvA gene, are referred as «reference 1» and «reference 2», respectively, and in MM_VAB20 medium condition, the valine productivity and the valine yield of strain 1, carrying 1 copy of ilvA gene, are referred as «reference 3» and «reference 4», respectively. The symbol «≈» indicates an increase lesser than 10%, the symbol «++» indicates an increase between 30 and 100%, and the symbol «+++» indicates an increase greater than 100%, compared to appropriate reference.

As shown in Table 2, the strain 2 carrying 2 copies of ilvA gene has an improved valine productivity whatever the medium used compared to strain 1. And the strain 3 carrying more than 2 copies of ilvA gene has an improved valine productivity and yield particularly in MM_VAB20 medium condition.

These results are surprising as in valine producing strains described in literature, the ilvA gene is often deleted or attenuated.

Improvement of Valine Production Related to ilvA Gene Copies into Other Valine Producing Strain Backgrounds

The beneficial effect of ilvA copies was demonstrated into other genetic backgrounds, more precisely into:
the W3110 E. coli strain described as “VAMF (pKBRilvBNmutCED, pTrc184ygaZHIrp)” in Park et al, 2011, and in which the ilvA gene is deleted, and
the W3110 E. coli strain described as “VHY18” in Hao et al, 2020 and in which no modification of ilvA gene was made, so this strain carries one copy of ilvA gene.

In aim to increase the copy number of ilvA gene up to two into both strains, an additional copy of ilvA gene and its promoter was added into a locus cited in Application US2012/0252077 into both strains and the original ilvA gene was reconstructed at the endogenous locus into Park's strain, according protocols 1 and 2.

The Park's and Hao's strains and the equivalent strains with 2 copies of ilvA gene were cultivated as described into respective conditions described in Park et al, 2011 and Hao et al, 2020 and valine production was evaluated as described in protocol 4.

Into Park's strain background, the change from zero to two copies of ilvA gene slightly improves productivity, whereas in Hao's strain background, the change from one to two copies of ilvA gene improves productivity and yield in the same order of magnitude when compared to strain 2 described in example 1.

Moreover, the reconstruction of ilvA gene into Park's strain is economically beneficial due to the unnecessary addition of leucine and isoleucine to the culture medium.

### Example 2: Punctual Mutation in argP Gene Improves Valine Performance of Valine Producing Strain Carrying 2 Copies of ilvA Gene

### Construction of Strain 4

To construct the strain 4, wildtype argP allele (SEQ ID NO: 3) coding for a DNA-binding transcriptional dual regulator, was replaced by argP* allele (SEQ ID NO: 5) coding for ArgP mutant having amino acid substitution glutamate into aspartate at position 128 (SEQ ID NO: 6) into strain 2, according protocols 1 and 2.

Improvement of Valine Production of Strain 2 Owing argP* Allele

Strains 2 and 4 were grown according to protocol 4. Valine productivity and yield were measured.

TABLE 3
Valine productivity and yield of strains carrying or not
mutation in argP gene, besides 2 copies of ilvA gene.

| Culture medium | MM_VAB10 | | MM_VAB20 | |
|---|---|---|---|---|
| Strain | 2 | 4 | 2 | 4 |
| Gene argP | wildtype | Mutated | wildtype | mutated |
| ilvA copy number | 2 | 2 | 2 | 2 |
| Productivity | Reference 5 | +++ | Reference 7 | ++ |
| (g/L/h) | | | | |
| Yield | Reference 6 | ≈ | Reference 8 | ≈ |
| (g/g) | | | | |

In MM_VAB10 medium condition, the valine productivity and the valine yield of strain 2, carrying wildtype version of argP gene, are referred as « reference 5 » and « reference 6 », respectively, and in MM_VAB20 medium condition, the valine productivity and the valine yield of strain 2 are referred as « reference 7 » and « reference 8 », respectively. The symbol « ≈ » indicates an increase lesser than 10%, the symbol « ++ » indicates an increase between 30 and 100% and the symbol « +++ » indicates an increase greater than 100%, compared to appropriate reference.

In MM_VAB10 medium condition, the valine productivity and the valine yield of strain 2, carrying wildtype version of argP gene, are referred as «reference 5» and «reference 6», respectively, and in MM_VAB20 medium condition, the valine productivity and the valine yield of strain 2 are referred as «reference 7 » and «reference 8», respectively. The symbol «≈» indicates an increase lesser than 10%, the symbol «++» indicates an increase between 30 and 100% and the symbol «+++» indicates an increase greater than 100%, compared to appropriate reference.

As shown in Table 3, the strain 4 carrying mutation in argP gene (and 2 copies of ilvA gene) has an improved valine productivity whatever the medium used compared to strain 2 carrying wildtype allele of argP gene. The mutation of argP does not affect the yield of valine.

### Example 3: Punctual Mutation in Promotor of fepA Gene Improves Valine Performance of Valine Producing Strain Carrying 2 Copies of ilvA Gene

### Construction of Strain 5

The gene fepA (SEQ ID NO: 82) codes for FepA protein (SEQ ID NO: 83), a ferric enterobactin outer membrane transporter. To construct the strain 5, wildtype fepA promoter sequence was replaced by mutated one into strain 2, according protocols 1 and 2. The mutated promoter possesses a nucleic acid base substitution T into C at −3 position from transcription start (SEQ ID NO: 8).

Improvement of Valine Production of Strain 2 Owing Mutation in fepA Promoter

Strains 2 and 5 were grown according to protocol 4. Valine productivity and yield were measured.

TABLE 4
Valine productivity and yield of strains carrying or not mutation
in fepA promoter, besides 2 copies of ilvA gene.

| Culture medium | MM_VAB10 | | MM_VAB20 | |
|---|---|---|---|---|
| Strain | 2 | 5 | 2 | 5 |
| Promoter fepA | wildtype | mutated | wildtype | mutated |
| gene | | | | |
| ilvA copy number | 2 | 2 | 2 | 2 |
| Productivity | Reference 5 | ≈ | Reference 7 | ++ |
| (g/L/h) | | | | |
| Yield | Reference 6 | ≈ | Reference 8 | ≈ |
| (g/g) | | | | |

In MM_VAB10 medium condition, the valine productivity and the valine yield of strain 2, carrying wildtype version of fepA promoter, are referred as « reference 5 » and « reference 6 », respectively, and in MM_VAB20 medium condition, the valine productivity and the valine yield of strain 2 are referred as « reference 7 » and « reference 8 », respectively. The symbol « ≈ » indicates an increase lesser than 10% and « ++ » an increase between 30 and 100%, compared to appropriate reference.

In MM_VAB10 medium condition, the valine productivity and the valine yield of strain 2, carrying wildtype version of fepA promoter, are referred as «reference 5» and «reference 6», respectively, and in MM_VAB20 medium condition, the valine productivity and the valine yield of strain 2 are referred as «reference 7» and «reference 8», respectively. The symbol «≈» indicates an increase lesser than 10% and «++» an increase between 30 and 100%, compared to appropriate reference.

As shown in Table 4, the strain 5 carrying mutation in fepA promoter (and 2 copies of ilvA gene) has an improved valine productivity in MM_VAB20 medium condition compared to strain 2 carrying wildtype sequence of fepA promoter. The mutation of fepA promoter does not affect the yield of valine.

### Example 4: Punctual Mutation in fepA Promoter Improves Valine Performance of Valine Producing Strain Carrying 2 Copies of ilvA Gene and a Mutation in argP Gene

### Construction of Strain 6

To construct the strain 6, wildtype fepA promoter sequence was replaced by mutated one into strain 4, according protocols 1 and 2. The mutated promoter possesses a nucleic acid base substitution T into C at −3 position from transcription start (SEQ ID NO: 8).

Improvement of Valine Production of Strain 4 Owing Mutation in fepA Promoter

Strains 4 and 6 were grown according to protocol 4. Valine productivity and yield were measured.

TABLE 5
Valine productivity and yield of strains carrying or not mutation in
fepA promoter, besides 2 copies of ilvA gene and mutation in argP gene.

| Culture medium | MM_VAB10 | | MM_VAB20 | |
|---|---|---|---|---|
| Strain | 4 | 6 | 4 | 6 |
| Gene argP | mutated | mutated | mutated | mutated |
| Promoter fepA gene | wildtype | mutated | wildtype | mutated |
| ilvA copy number | 2 | 2 | 2 | 2 |
| Productivity | Reference 9 | ≈ | Reference 11 | + |
| (g/L/h) | | | | |
| Yield | Reference 10 | ≈ | Reference 12 | ≈ |
| (g/g) | | | | |

In MM_VAB10 medium condition, the valine productivity and the valine yield of strain 4, carrying mutated allele of argP and wildtype sequence of fepA promoter, are referred as « reference 9 » and « reference 10 », respectively, and in MM_VAB20 medium condition, the valine productivity and the valine yield of strain 4 are referred as « reference 11 » and « reference 12 », respectively. The symbol « ≈ » indicates an increase lesser than 10% and « + » an increase between 10 and 30%, compared to appropriate reference.

In MM_VAB10 medium condition, the valine productivity and the valine yield of strain 4, carrying mutated allele of argP and wildtype sequence of fepA promoter, are referred as «reference 9» and «reference 10 », respectively, and in MM_VAB20 medium condition, the valine productivity and the valine yield of strain 4 are referred as «reference 11 » and «reference 12 », respectively. The symbol «≈» indicates an increase lesser than 10% and «+» an increase between 10 and 30%, compared to appropriate reference.

As shown in Table 5, the strain 6 carrying mutation in fepA promoter (and 2 copies of ilvA gene and the mutation in argP gene) has an improved valine productivity in MM_VAB20 medium condition compared to strain 4 carrying wildtype sequence of fepA promoter. The mutation of fepA promoter does not affect the yield of valine.

### REFERENCES

Bantscheff et al., (2007), Analytical and Bioanalytical Chemistry, vol. 389 (4): 1017-1031.
Bartolomé B, Jubete Y, Martínez E, de la Cruz F et al., (1991), Gene., 1991 Jun. 15; 102 (1): 75-8.
Burnette, (1981), Analytical Biochemistry, 112 (2): 195-203.
Carrier T & Keasling J., (1999), Biotechnol Prog., 15 (1): 58-64. Construction and properties of a family of pACYC184-derived cloning vectors compatible with pBR322 and its derivatives.
Datsenko and Wanner, (2000), Proc Natl Acad Sci USA., 97:6640-6645.
Davis & Olsen., (2011), Mol. Biol. Evol., 28 (1): 211-221.
Deml et al., (2011), J. Virol., 75 (22): 10991-11001.
Engvall and Perlman (1981), Immunochemistry, 8:871-874. Fed-batch culture of Escherichia coli for L-valine production based on in silico flux response analysis.
Graf et al., (2000), J. Virol., 74 (22): 10/22-10826.
Hao et al., (2020), Y, Ma Q, Liu X, Fan X, Men J, Wu H, Jiang S, Tian D, Xiong B, Xie X. Metab Eng., 2020 November; 62:198-206. High-yield production of L-valine in engineered Escherichia coli by a novel two-stage fermentation.
Leuchtenberger, et al, (2005) Appl. Microbiol. Biotechnol. 69, 1-8
Needleman and Wunsch (1970), J. Mol. Biol., 48 (3), 443-453.
Park et al., (2012), ACS synthetic biology, 1 (11): 532-540
Park J H, Kim T Y, Lee K H, Lee S Y et al., (2011), Biotechnol Bioeng., 2011 April; 108 (4): 934-46.
Salis H., (2011), Methods Enzymol., 498:19-42
Segel I., (1993), Enzyme kinetics, John Wiley & Sons, pp. 44-54 and 100-112

1. Microorganism genetically modified for the production of valine, wherein the microorganism overexpresses a ilvA gene coding a threonine deaminase and/or exhibits an increased threonine deaminase activity as compared to the expression level and/or threonine deaminase activity in a corresponding wild-type microorganism, and wherein the microorganism further comprises a mutated argP gene coding DNA-binding transcriptional dual regulator.

2. The microorganism of claim 1, wherein the ilvA gene is overexpressed in the microorganism such as by modifying the promoter regulating the expression of the ilvA gene, by increasing the number of copies of the ilvA gene present in the microorganism, or by overexpressing the ilvA gene from a plasmid, by improving stability of the ilvA mRNA or increasing IlvA protein quantity by optimization of Ribosome Binding Site.

3. The microorganism of claim 1 or 2, wherein the ilvA gene is overexpressed in the microorganism by increasing the number of copies of the ilvA gene present in the microorganism, preferably leading to at least two copies of the gene, more preferably leading to two copies of the gene.

4. The microorganism of any of claims 1 to 3, wherein the mutated argP gene is modified so as to lead a substitution of the amino acid at position 128, preferably a substitution of Glu by Asp.

5. The microorganism of any of claims 1 to 4, wherein the microorganism further overexpresses a fepA gene coding ferric enterobactin outer membrane transporter and/or exhibits an increased ferric enterobactin outer membrane transporter activity as compared to the expression level and/or ferric enterobactin outer membrane transporter activity in a corresponding wild-type microorganism.

6. The microorganism of claim 5, wherein the fepA gene is overexpressed by genetic modification, such as by modifying the promoter regulating the expression of the fepA gene, by increasing the number of copies of the fepA gene present in the microorganism, or by overexpressing the fepA gene from a plasmid, by improving stability of the fepA mRNA or increasing FepA protein quantity by optimization of Ribosome Binding Site, preferably by mutating the promoter regulating the expression of the fepA gene.

7. The microorganism of claim 6, wherein the fepA gene is overexpressed by at least one base replacement in the promoter sequence regulating the expression of the fepA gene.

8. The microorganism of any of claims 1 to 7, further comprising a deletion of at least one gene selected from the group consisting of IdhA, adhE and mgsA.

9. The microorganism of any of claims 1 to 8, further comprising overexpression of at least one gene selected from the group consisting of vdh, ilvD, ilvC, ilvB, and ilvN*.

10. The microorganism of any of claims 1 to 9, wherein said microorganism belongs to the family of bacteria Enterobacteriaceae, Corynebacteriaceae, Bacillaceae, Streptococcae or Lactobacillus, or to the family of fungus such as Hemiascomycetus, filamentous fungus or yeast.

11. The microorganism of claim 10, wherein said Enterobacteriaceae bacterium is Escherichia coli, said Corynebacteriaceae bacterium is Corynebacterium glutamicum or said Bacillaceae is Bacillus subtilis said Streptococcae is Streptococcus thermophiles, said Lactobacillus is Lactobacillus lactis, said Hemiascomycetus yeast is Saccharomyces cerevisiae or Yarrowia lipolytica and said filamentous fungus is Tricchoderma rezeii or Aspergillus niger, preferably wherein said microorganism is Escherichia coli.

12. The microorganism of claim 1, with the number CNCM I-5911, deposited on Oct. 19, 2022 at the Collection Nationale de Cultures de Microorganismes, Pasteur Institute, 25 Rue du Docteur Roux, 75724 PARIS Cedex 15, FRANCE.

13. Method for the production of valine comprising the steps of:
a) culturing a microorganism genetically modified for the production of valine according to any of claims 1 to 12 in an appropriate culture medium comprising a source of carbon, and
b) recovering valine from the culture medium.

14. The method of claim 13, wherein the recovering of valine comprises at least the steps of:
a) clarification of the fermentation medium for removing the insoluble organic impurities,
b) treatment of the product of the preceding step on an adsorbent such as activated charcoal for removing soluble organic impurities and inorganic impurities,
c) evaporation of water and crystallization of the product obtained,
d) recovering valine.


SEQUENCE LISTING (83 sequences)

SEQ ID NO: 1 - DNA, 1545 bp, Escherichia coli
  source 1..1545: mol_type = genomic DNA
atggctgactcgcaacccctgtccggtgctccggaaggtgccgaatatttaagagcagtgctgcgcgcgccggtttacgaggcggcgcaggttacgccgctacaaaaaatggaaaaactgtcgtcgcgtcttgataacgtcattctggtgaagcgcgaagatcgccagccagtgcacagctttaagctgcgcggcgcatacgccatgatggcgggcctgacggaagaacagaaagcgcacggcgtgatcactgcttctgcgggtaaccacgcgcagggcgtcgcgttttcttctgcgcggttaggcgtgaaggccctgatcgttatgccaaccgccaccgccgacatcaaagtcgacgcggtgcgcggcttcggcggcgaagtgctgctccacggcgcgaactttgatgaagcgaaagccaaagcgatcgaactgtcacagcagcaggggttcacctgggtgccgccgttcgaccatccgatggtgattgccgggcaaggcacgctggcgctggaactgctccagcaggacgcccatctcgaccgcgtatttgtgccagtcggcggcggcggtctggctgctggcgtggcggtgctgatcaaacaactgatgccgcaaatcaaagtgatcgccgtagaagcggaagactccgcctgcctgaaagcagcgctggatgcgggtcatccggttgatctgccgcgcgtagggctatttgctgaaggcgtagcggtaaaacgcatcggtgacgaaaccttccgtttatgccaggagtatctcgacgacatcatcaccgtcgatagcgatgcgatctgtgcggcgatgaaggatttattcgaagatgtgcgcgcggtggcggaaccctctggcgcgctggcgctggcgggaatgaaaaaatatatcgccctgcacaacattcgcggcgaacggctggcgcatattctttccggtgccaacgtgaacttccacggcctgcgctacgtctcagaacgctgcgaactgggcgaacagcgtgaagcgttgttggcggtgaccattccggaagaaaaaggcagcttcctcaaattctgccaactgcttggcgggcgttcggtcaccgagttcaactaccgttttgccgatgccaaaaacgcctgcatctttgtcggtgtgcgcctgagccgcggcctcgaagagcgcaaagaaattttgcagatgctcaacgacggcggctacagcgtggttgatctctccgacgacgaaatggcgaagctacacgtgcgctatatggtcggcggacgtccatcgcatccgttgcaggaacgcctctacagcttcgaattcccggaatcaccgggcgcgctgctgcgcttcctcaacacgctgggtacgtactggaacatttctttgttccactatcgcagccatggcaccgactacgggcgcgtactggcggcgttcgaacttggcgaccatgaaccggatttcgaaacccggctgaatgagctgggctacgattgccacgacgaaaccaataacccggcgttcaggttctttttggcgggttag

SEQ ID NO: 2 - protein, 514 aa, Escherichia coli [sequence omitted]

SEQ ID NO: 3 - DNA, 894 bp, Escherichia coli
  source 1..894: mol_type = genomic DNA
atgaaacgcccggactacagaacattacaggcactggatgcggtgatacgtgaacgaggatttgagcgcgcggcacaaaagctgtgcattacacaatcagccgtctcacagcgcattaagcaactggaaaatatgttcgggcagccgctgttggtgcgtaccgtaccgccgcgcccgacggaacaagggcaaaaactgctggcactgctgcgccaggtggagttgctggaagaagagtggctgggcgatgaacaaaccggttcgactccgctgctgctttcactggcggtcaacgccgacagtctggcgacgtggttgcttcctgcactggctcctgtgttggctgattcgcctatccgcctcaacttgcaggtagaagatgaaacccgcactcaggaacgtctgcgccgcggcgaagtggtcggcgcggtgagtattcaacatcaggcgctgccgagttgtcttgtcgataaacttggtgcgctcgactatctgttcgtcagctcaaaaccctttgccgaaaaatatttccctaacggcgtaacgcgttcggcattactgaaagcgccagtggtcgcgtttgaccatcttgacgatatgcaccaggcctttttgcagcaaaacttcgatctgcctccaggcagcgtgccctgccatatcgttaattcttcagaagcgttcgtacaacttgctcgccagggcaccacctgctgtatgatcccgcacctgcaaatcgagaaagagctggccagcggtgaactgattgacttaacgcctgggctatttcaacgacggatgctctactggcaccgctttgctcctgaaagccgcatgatgcgtaaagtcactgatgcgttactcgattatggtcacaaagtccttcgtcaggattaa

SEQ ID NO: 4 - protein, 297 aa, Escherichia coli
MKRPDYRTLQALDAVIRERGFERAAQKLCITQSAVSQRIKQLENMFGQPLLVRTVPPRPTEQGQKLLALLRQVELLEEEWLGDEQTGSTPLLLSLAVNADSLATWLLPALAPVLADSPIRLNLQVEDETRTQERLRRGEVVGAVSIQHQALPSCLVDKLGALDYLFVSSKPFAEKYFPNGVTRSALLKAPVVAFDHLDDMHQAFLQQNFDLPPGSVPCHIVNSSEAFVQLARQGTTCCMIPHLQIEKELASGELIDLTPGLFQRRMLYWHRFAPESRMMRKVTDALLDYGHKVLRQD

SEQ ID NO: 5 - DNA, 894 bp, Escherichia coli
  source 1..894: mol_type = other DNA
atgaaacgcccggactacagaacattacaggcactggatgcggtgatacgtgaacgaggatttgagcgcgcggcacaaaagctgtgcattacacaatcagccgtctcacagcgcattaagcaactggaaaatatgttcgggcagccgctgttggtgcgtaccgtaccgccgcgcccgacggaacaagggcaaaaactgctggcactgctgcgccaggtggagttgctggaagaagagtggctgggcgatgaacaaaccggttcgactccgctgctgctttcactggcggtcaacgccgacagtctggcgacgtggttgcttcctgcactggctcctgtgttggctgattcgcctatccgcctcaacttgcaggtagaagatgatacccgcactcaggaacgtctgcgccgcggcgaagtggtcggcgcggtgagtattcaacatcaggcgctgccgagttgtcttgtcgataaacttggtgcgctcgactatctgttcgtcagctcaaaaccctttgccgaaaaatatttccctaacggcgtaacgcgttcggcattactgaaagcgccagtggtcgcgtttgaccatcttgacgatatgcaccaggcctttttgcagcaaaacttcgatctgcctccaggcagcgtgccctgccatatcgttaattcttcagaagcgttcgtacaacttgctcgccagggcaccacctgctgtatgatcccgcacctgcaaatcgagaaagagctggccagcggtgaactgattgacttaacgcctgggctatttcaacgacggatgctctactggcaccgctttgctcctgaaagccgcatgatgcgtaaagtcactgatgcgttactcgattatggtcacaaagtccttcgtcaggattaa

SEQ ID NO: 6 - protein, 297 aa, Escherichia coli
MKRPDYRTLQALDAVIRERGFERAAQKLCITQSAVSQRIKQLENMFGQPLLVRTVPPRPTEQGQKLLALLRQVELLEEEWLGDEQTGSTPLLLSLAVNADSLATWLLPALAPVLADSPIRLNLQVEDDTRTQERLRRGEVVGAVSIQHQALPSCLVDKLGALDYLFVSSKPFAEKYFPNGVTRSALLKAPVVAFDHLDDMHQAFLQQNFDLPPGSVPCHIVNSSEAFVQLARQGTTCCMIPHLQIEKELASGELIDLTPGLFQRRMLYWHRFAPESRMMRKVTDALLDYGHKVLRQD

SEQ ID NO: 7 - DNA, 81 bp, Escherichia coli
  source 1..81: mol_type = genomic DNA
ccatgtttactgtgcaatttttcattgattgcagaaatatattgataatattattgataactatttgcatttgcaatagcg

SEQ ID NO: 8 - DNA, 81 bp, Escherichia coli
  source 1..81: mol_type = other DNA
ccatgtttactgtgcaatttttcattgattgcagaaatatattgataatattattgacaactatttgcatttgcaatagcg

SEQ ID NO: 9 - DNA, 990 bp, Escherichia coli
  source 1..990: mol_type = genomic DNA
atgaaactcgccgtttatagcacaaaacagtacgacaagaagtacctgcaacaggtgaacgagtcctttggctttgagctggaattttttgactttctgctgacggaaaaaaccgctaaaactgccaatggctgcgaagcggtatgtattttcgtaaacgatgacggcagccgcccggtgctggaagagctgaaaaagcacggcgttaaatatatcgccctgcgctgtgccggtttcaataacgtcgaccttgacgcggcaaaagaactggggctgaaagtagtccgtgttccagcctatgatccagaggccgttgctgaacacgccatcggtatgatgatgacgctgaaccgccgtattcaccgcgcgtatcagcgtacccgtgatgctaacttctctctggaaggtctgaccggctttactatgtatggcaaaacggcaggcgttatcggtaccggtaaaatcggtgtggcgatgctgcgcattctgaaaggttttggtatgcgtctgctggcgttcgatccgtatccaagtgcagcggcgctggaactcggtgtggagtatgtcgatctgccaaccctgttctctgaatcagacgttatctctctgcactgcccgctgacaccggaaaactatcatctgttgaacgaagccgccttcgaacagatgaaaaatggcgtgatgatcgtcaataccagtcgcggtgcattgattgattctcaggcagcaattgaagcgctgaaaaatcagaaaattggttcgttgggtatggacgtgtatgagaacgaacgcgatctattctttgaagataaatccaacgacgtgatccaggatgacgtattccgtcgcctgtctgcctgccacaacgtgctgtttaccgggcaccaggcattcctgacagcagaagctctgaccagtatttctcagactacgctgcaaaacttaagcaatctggaaaaaggcgaaacctgcccgaacgaactggtttaa

SEQ ID NO: 10 - protein, 329 aa, Escherichia coli
MKLAVYSTKQYDKKYLQQVNESFGFELEFFDFLLTEKTAKTANGCEAVCIFVNDDGSRPVLEELKKHGVKYIALRCAGFNNVDLDAAKELGLKVVRVPAYDPEAVAEHAIGMMMTLNRRIHRAYQRTRDANFSLEGLTGFTMYGKTAGVIGTGKIGVAMLRILKGFGMRLLAFDPYPSAAALELGVEYVDLPTLFSESDVISLHCPLTPENYHLLNEAAFEQMKNGVMIVNTSRGALIDSQAAIEALKNQKIGSLGMDVYENERDLFFEDKSNDVIQDDVFRRLSACHNVLFTGHQAFLTAEALTSISQTTLQNLSNLEKGETCPNELV

SEQ ID NO: 11 - DNA, 2676 bp, Escherichia coli
  source 1..2676: mol_type = genomic DNA
atggctgttactaatgtcgctgaacttaacgcactcgtagagcgtgtaaaaaaagcccagcgtgaatatgccagtttcactcaagagcaagtagacaaaatcttccgcgccgccgctctggctgctgcagatgctcgaatcccactcgcgaaaatggccgttgccgaatccggcatgggtatcgtcgaagataaagtgatcaaaaaccactttgcttctgaatatatctacaacgcctataaagatgaaaaaacctgtggtgttctgtctgaagacgacacttttggtaccatcactatcgctgaaccaatcggtattatttgcggtatcgttccgaccactaacccgacttcaactgctatcttcaaatcgctgatcagtctgaagacccgtaacgccattatcttctccccgcacccgcgtgcaaaagatgccaccaacaaagcggctgatatcgttctgcaggctgctatcgctgccggtgctccgaaagatctgatcggctggatcgatcaaccttctgttgaactgtctaacgcactgatgcaccacccagacatcaacctgatcctcgcgactggtggtccgggcatggttaaagccgcatacagctccggtaaaccagctatcggtgtaggcgcgggcaacactccagttgttatcgatgaaactgctgatatcaaacgtgcagttgcatctgtactgatgtccaaaaccttcgacaacggcgtaatctgtgcttctgaacagtctgttgttgttgttgactctgtttatgacgctgtacgtgaacgttttgcaacccacggcggctatctgttgcagggtaaagagctgaaagctgttcaggatgttatcctgaaaaacggtgcgctgaacgcggctatcgttggtcagccagcctataaaattgctgaactggcaggcttctctgtaccagaaaacaccaagattctgatcggtgaagtgaccgttgttgatgaaagcgaaccgttcgcacatgaaaaactgtccccgactctggcaatgtaccgcgctaaagatttcgaagacgcggtagaaaaagcagagaaactggttgctatgggcggtatcggtcatacctcttgcctgtacactgaccaggataaccaaccggctcgcgtttcttacttcggtcagaaaatgaaaacggcgcgtatcctgattaacaccccagcgtctcagggtggtatcggtgacctgtataacttcaaactcgcaccttccctgactctgggttgtggttcttggggtggtaactccatctctgaaaacgttggtccgaaacacctgatcaacaagaaaaccgttgctaagcgagctgaaaacatgttgtggcacaaacttccgaaatctatctacttccgccgtggctccctgccaatcgcgctggatgaagtgattactgatggccacaaacgtgcgctcatcgtgactgaccgcttcctgttcaacaatggttatgctgatcagatcacttccgtactgaaagcagcaggcgttgaaactgaagtcttcttcgaagtagaagcggacccgaccctgagcatcgttcgtaaaggtgcagaactggcaaactccttcaaaccagacgtgattatcgcgctgggtggtggttccccgatggacgccgcgaagatcatgtgggttatgtacgaacatccggaaactcacttcgaagagctggcgctgcgctttatggatatccgtaaacgtatctacaagttcccgaaaatgggcgtgaaagcgaaaatgatcgctgtcaccaccacttctggtacaggttctgaagtcactccgtttgcggttgtaactgacgacgctactggtcagaaatatccgctggcagactatgcgctgactccggatatggcgattgtcgacgccaacctggttatggacatgccgaagtccctgtgtgctttcggtggtctggacgcagtaactcacgccatggaagcttatgtttctgtactggcatctgagttctctgatggtcaggctctgcaggcactgaaactgctgaaagaatatctgccagcgtcctaccacgaagggtctaaaaatccggtagcgcgtgaacgtgttcacagtgcagcgactatcgcgggtatcgcgtttgcgaacgccttcctgggtgtatgtcactcaatggcgcacaaactgggttcccagttccatattccgcacggtctggcaaacgccctgctgatttgtaacgttattcgctacaatgcgaacgacaacccgaccaagcagactgcattcagccagtatgaccgtccgcaggctcgccgtcgttatgctgaaattgccgaccacttgggtctgagcgcaccgggcgaccgtactgctgctaagatcgagaaactgctggcatggctggaaacgctgaaagctgaactgggtattccgaaatctatccgtgaagctggcgttcaggaagcagacttcctggcgaacgtggataaactgtctgaagatgcattcgatgaccagtgcaccggcgctaacccgcgttacccgctgatctccgagctgaaacagattctgctggatacctactacggtcgtgattatgtagaaggtgaaactgcagcgaagaaagaagctgctccggctaaagctgagaaaaaagcgaaaaaatccgcttaa

SEQ ID NO: 12 - protein, 891 aa, Escherichia coli
MAVTNVAELNALVERVKKAQREYASFTQEQVDKIFRAAALAAADARIPLAKMAVAESGMGIVEDKVIKNHFASEYIYNAYKDEKTCGVLSEDDTFGTITIAEPIGIICGIVPTTNPTSTAIFKSLISLKTRNAIIFSPHPRAKDATNKAADIVLQAAIAAGAPKDLIGWIDQPSVELSNALMHHPDINLILATGGPGMVKAAYSSGKPAIGVGAGNTPVVIDETADIKRAVASVLMSKTFDNGVICASEQSVVVVDSVYDAVRERFATHGGYLLQGKELKAVQDVILKNGALNAAIVGQPAYKIAELAGFSVPENTKILIGEVTVVDESEPFAHEKLSPTLAMYRAKDFEDAVEKAEKLVAMGGIGHTSCLYTDQDNQPARVSYFGQKMKTARILINTPASQGGIGDLYNFKLAPSLTLGCGSWGGNSISENVGPKHLINKKTVAKRAENMLWHKLPKSIYFRRGSLPIALDEVITDGHKRALIVTDRFLFNNGYADQITSVLKAAGVETEVFFEVEADPTLSIVRKGAELANSFKPDVIIALGGGSPMDAAKIMWVMYEHPETHFEELALRFMDIRKRIYKFPKMGVKAKMIAVTTTSGTGSEVTPFAVVTDDATGQKYPLADYALTPDMAIVDANLVMDMPKSLCAFGGLDAVTHAMEAYVSVLASEFSDGQALQALKLLKEYLPASYHEGSKNPVARERVHSAATIAGIAFANAFLGVCHSMAHKLGSQFHIPHGLANALLICNVIRYNANDNPTKQTAFSQYDRPQARRRYAEIADHLGLSAPGDRTAAKIEKLLAWLETLKAELGIPKSIREAGVQEADFLANVDKLSEDAFDDQCTGANPRYPLISELKQILLDTYYGRDYVEGETAAKKEAAPAKAEKKAKKSA

SEQ ID NO: 13 - DNA, 459 bp, Escherichia coli
  source 1..459: mol_type = genomic DNA
atggaactgacgactcgcactttacctgcgcggaaacatattgcgctggtggcacacgatcactgcaaacaaatgctgatgagctgggtggaacggcatcaaccgttactggaacaacacgtactgtatgcaacaggcactaccggtaacttaatttcccgcgcgaccggcatgaacgtcaacgcgatgttgagtggcccaatggggggtgaccagcaggttggcgcattgatctcagaagggaaaattgatgtattgattttcttctgggatccactaaatgccgtgccgcacgatcctgacgtgaaagccttgctgcgtctggcgacggtatggaacattccggtcgccaccaacgtggcaacggcagacttcataatccagtcgccgcatttcaacgacgcggtcgatattctgatccccgattatcagcgttatctcgcggaccgtctgaagtaa

SEQ ID NO: 14 - protein, 152 aa, Escherichia coli [sequence omitted]

SEQ ID NO: 15 - DNA, 1809 bp, Escherichia coli
  source 1..1809: mol_type = genomic DNA
gtgcaaacctttcaagccgatcttgccattgtaggcgccggtggcgcgggattacgtgctgcaattgctgccgcgcaggcaaatccgaatgcaaaaatcgcactaatctcaaaagtatacccgatgcgtagccataccgttgctgcagaagggggctccgccgctgtcgcgcaggatcatgacagcttcgaatatcactttcacgatacagtagcgggtggcgactggttgtgtgagcaggatgtcgtggattatttcgtccaccactgcccaaccgaaatgacccaactggaactgtggggatgcccatggagccgtcgcccggatggtagcgtcaacgtacgtcgcttcggcggcatgaaaatcgagcgcacctggttcgccgccgataagaccggcttccatatgctgcacacgctgttccagacctctctgcaattcccgcagatccagcgttttgacgaacatttcgtgctggatattctggttgatgatggtcatgttcgcggcctggtagcaatgaacatgatggaaggcacgctggtgcagatccgtgctaacgcggtcgttatggctactggcggtgcgggtcgcgtttatcgttacaacaccaacggcggcatcgttaccggtgacggtatgggtatggcgctaagccacggcgttccgctgcgtgacatggaattcgttcagtatcacccaaccggtctgccaggttccggtatcctgatgaccgaaggttgccgcggtgaaggcggtattctggtcaacaaaaatggctaccgttatctgcaagattacggcatgggcccggaaactccgctgggcgagccgaaaaacaaatatatggaactgggtccacgcgacaaagtctctcaggccttctggcacgaatggcgtaaaggcaacaccatctccacgccgcgtggcgatgtggtttatctcgacttgcgtcacctcggcgagaaaaaactgcatgaacgtctgccgttcatctgcgaactggcgaaagcgtacgttggcgtcgatccggttaaagaaccgattccggtacgtccgaccgcacactacaccatgggcggtatcgaaaccgatcagaactgtgaaacccgcattaaaggtctgttcgccgtgggtgaatgttcctctgttggtctgcacggtgcaaaccgtctgggttctaactccctggcggaactggtggtcttcggccgtctggccggtgaacaagcgacagagcgtgcagcaactgccggtaatggcaacgaagcggcaattgaagcgcaggcagctggcgttgaacaacgtctgaaagatctggttaaccaggatggcggcgaaaactgggcgaagatccgcgacgaaatgggcctggctatggaagaaggctgcggtatctaccgtacgccggaactgatgcagaaaaccatcgacaagctggcagagctgcaggaacgcttcaagcgcgtgcgcatcaccgacacttccagcgtgttcaacaccgacctgctctacaccattgaactgggccacggtctgaacgttgctgaatgtatggcgcactccgcaatggcacgtaaagagtcccgcggcgcgcaccagcgtctggacgaaggttgcaccgagcgtgacgacgtcaacttcctcaaacacaccctcgccttccgcgatgctgatggcacgactcgcctggagtacagcgacgtgaagattactacgctgccgccagctaaacgcgtttacggtggcgaagcggatgcagccgataaggcggaagcagccaataagaaggagaaggcgaatggctga

SEQ ID NO: 16 - protein, 602 aa, Escherichia coli
VQTFQADLAIVGAGGAGLRAAIAAAQANPNAKIALISKVYPMRSHTVAAEGGSAAVAQDHDSFEYHFHDTVAGGDWLCEQDVVDYFVHHCPTEMTQLELWGCPWSRRPDGSVNVRRFGGMKIERTWFAADKTGFHMLHTLFQTSLQFPQIQRFDEHFVLDILVDDGHVRGLVAMNMMEGTLVQIRANAVVMATGGAGRVYRYNTNGGIVTGDGMGMALSHGVPLRDMEFVQYHPTGLPGSGILMTEGCRGEGGILVNKNGYRYLQDYGMGPETPLGEPKNKYMELGPRDKVSQAFWHEWRKGNTISTPRGDVVYLDLRHLGEKKLHERLPFICELAKAYVGVDPVKEPIPVRPTAHYTMGGIETDQNCETRIKGLFAVGECSSVGLHGANRLGSNSLAELVVFGRLAGEQATERAATAGNGNEAAIEAQAAGVEQRLKDLVNQDGGENWAKIRDEMGLAMEEGCGIYRTPELMQKTIDKLAELQERFKRVRITDTSSVFNTDLLYTIELGHGLNVAECMAHSAMARKESRGAHQRLDEGCTERDDVNFLKHTLAFRDADGTTRLEYSDVKITTLPPAKRVYGGEADAADKAEAANKKEKANG

SEQ ID NO: 17 - DNA, 735 bp, Escherichia coli
  source 1..735: mol_type = genomic DNA
atggctgagatgaaaaacctgaaaattgaggtggtgcgctataacccggaagtcgataccgcaccgcatagcgcattctatgaagtgccttatgacgcaactacctcattactggatgcgctgggctacatcaaagacaacctggcaccggacctgagctaccgctggtcctgccgtatggcgatttgtggttcctgcggcatgatggttaacaacgtgccaaaactggcatgtaaaaccttcctgcgtgattacaccgacggtatgaaggttgaagcgttagctaacttcccgattgaacgcgatctggtggtcgatatgacccacttcatcgaaagtctggaagcgatcaaaccgtacatcatcggcaactcccgcaccgcggatcagggtactaacatccagaccccggcgcagatggcgaagtatcaccagttctccggttgcatcaactgtggtttgtgctacgccgcgtgcccgcagtttggcctgaacccagagttcatcggtccggctgccattacgctggcgcatcgttataacgaagatagccgcgaccacggtaagaaggagcgtatggcgcagttgaacagccagaacggcgtatggagctgtactttcgtgggctactgctccgaagtctgcccgaaacacgtcgatccggctgcggccattcagcagggcaaagtagaaagttcgaaagactttcttatcgcgaccctgaaaccacgctaa

SEQ ID NO: 18 - protein, 244 aa, Escherichia coli
MAEMKNLKIEVVRYNPEVDTAPHSAFYEVPYDATTSLLDALGYIKDNLAPDLSYRWSCRMAICGSCGMMVNNVPKLACKTFLRDYTDGMKVEALANFPIERDLVVDMTHFIESLEAIKPYIIGNSRTADQGTNIQTPAQMAKYHQFSGCINCGLCYAACPQFGLNPEFIGPAAITLAHRYNEDSRDHGKKERMAQLNSQNGVWSCTFVGYCSEVCPKHVDPAAAIQQGKVESSKDFLIATLKPR

SEQ ID NO: 19 - DNA, 396 bp, Escherichia coli
  source 1..396: mol_type = genomic DNA
atgacgactaaacgtaaaccgtatgtacggccaatgacgtccacctggtggaaaaaattgccgttttatcgcttttacatgctgcgcgaaggcacggcggttccggctgtgtggttcagcattgaactgattttcgggctgtttgccctgaaaaatggcccggaagcctgggcgggattcgtcgactttttacaaaacccggttatcgtgatcattaacctgatcactctggcggcagctctgctgcacaccaaaacctggtttgaactggcaccgaaagcggccaatatcattgtaaaagacgaaaaaatgggaccagagccaattatcaaaagtctctgggcggtaactgtggttgccaccatcgtaatcctgtttgttgccctgtactggtaa

SEQ ID NO: 20 - protein, 131 aa, Escherichia coli
MTTKRKPYVRPMTSTWWKKLPFYRFYMLREGTAVPAVWFSIELIFGLFALKNGPEAWAGFVDFLQNPVIVIINLITLAAALLHTKTWFELAPKAANIIVKDEKMGPEPIIKSLWAVTVVATIVILFVALYW

SEQ ID NO: 21 - DNA, 360 bp, Escherichia coli
  source 1..360: mol_type = genomic DNA
atgattaatccaaatccaaagcgttctgacgaaccggtattctggggcctcttcggggccggtggtatgtggagcgccatcattgcgccggtgatgatcctgctggtgggtattctgctgccactggggttgtttccgggtgatgcgctgagctacgagcgcgttctggcgttcgcgcagagcttcattggtcgcgtattcctgttcctgatgatcgttctgccgctgtggtgtggtttacaccgtatgcaccacgcgatgcacgatctgaaaatccacgtacctgcgggcaaatgggttttctacggtctggctgctatcctgacagttgtcacgctgattggtgtcgttacaatctaa

SEQ ID NO: 22 - protein, 119 aa, Escherichia coli
MINPNPKRSDEPVFWGLFGAGGMWSAIIAPVMILLVGILLPLGLFPGDALSYERVLAFAQSFIGRVFLFLMIVLPLWCGLHRMHHAMHDLKIHVPAGKWVFYGLAAILTVVTLIGVVTI

SEQ ID NO: 23 - DNA, 741 bp, Escherichia coli
  source 1..741: mol_type = genomic DNA
atgtcagttattggtcgcattcactcctttgaatcctgtggaaccgtagacggcccaggtattcgctttatcacctttttccagggctgcctgatgcgctgcctgtattgtcataaccgcgacacctgggacacgcatggcggtaaagaagttaccgttgaagatttgatgaaggaagtggtgacctatcgccactttatgaacgcttccggcggcggcgttaccgcatccggcggtgaagcaatcctgcaagctgagtttgttcgtgactggttccgcgcctgcaaaaaagaaggcattcatacctgtctggacaccaacggttttgttcgtcgttacgatccggtgattgatgaactgctggaagtaaccgacctggtaatgctcgatctcaaacagatgaacgacgagatccaccaaaatctggttggagtttccaaccaccgcacgctggagttcgctaaatatctggcgaacaaaaatgtgaaggtgtggatccgctacgttgttgtcccaggctggtctgacgatgacgattcagcgcatcgcctcggtgaatttacccgtgatatgggcaacgttgagaaaatcgagcttctcccctaccacgagctgggcaaacacaaatgggtggcaatgggtgaagagtacaaactcgacggtgttaaaccaccgaagaaagagaccatggaacgcgtgaaaggcattcttgagcagtacggtcataaggtaatgttctaa

SEQ ID NO: 24 - protein, 246 aa, Escherichia coli
MSVIGRIHSFESCGTVDGPGIRFITFFQGCLMRCLYCHNRDTWDTHGGKEVTVEDLMKEVVTYRHFMNASGGGVTASGGEAILQAEFVRDWFRACKKEGIHTCLDTNGFVRRYDPVIDELLEVTDLVMLDLKQMNDEIHQNLVGVSNHRTLEFAKYLANKNVKVWIRYVVVPGWSDDDDSAHRLGEFTRDMGNVEKIELLPYHELGKHKWVAMGEEYKLDGVKPPKKETMERVKGILEQYGHKVMF

SEQ ID NO: 25 - DNA, 2283 bp, Escherichia coli
  source 1..2283: mol_type = genomic DNA
atgtccgagcttaatgaaaagttagccacagcctgggaaggttttaccaaaggtgactggcagaatgaagtaaacgtccgtgacttcattcagaaaaactacactccgtacgagggtgacgagtccttcctggctggcgctactgaagcgaccaccaccctgtgggacaaagtaatggaaggcgttaaactggaaaaccgcactcacgcgccagttgactttgacaccgctgttgcttccaccatcacctctcacgacgctggctacatcaacaagcagcttgagaaaatcgttggtctgcagactgaagctccgctgaaacgtgctcttatcccgttcggtggtatcaaaatgatcgaaggttcctgcaaagcgtacaaccgcgaactggatccgatgatcaaaaaaatcttcactgaataccgtaaaactcacaaccagggcgtgttcgacgtttacactccggacatcctgcgttgccgtaaatctggtgttctgaccggtctgccagatgcatatggccgtggccgtatcatcggtgactaccgtcgcgttgcgctgtacggtatcgactacctgatgaaagacaaactggcacagttcacttctctgcaggctgatctggaaaacggcgtaaacctggaacagactatccgtctgcgcgaagaaatcgctgaacagcaccgcgctctgggtcagatgaaagaaatggctgcgaaatacggctacgacatctctggtccggctaccaacgctcaggaagctatccagtggacttacttcggctacctggctgctgttaagtctcagaacggtgctgcaatgtccttcggtcgtacctccaccttcctggatgtgtacatcgaacgtgacctgaaagctggcaagatcaccgaacaagaagcgcaggaaatggttgaccacctggtcatgaaactgcgtatggttcgcttcctgcgtactccggaatacgatgaactgttctctggcgacccgatctgggcaaccgaatctatcggtggtatgggcctcgacggtcgtaccctggttaccaaaaacagcttccgtttcctgaacaccctgtacaccatgggtccgtctccggaaccgaacatgaccattctgtggtctgaaaaactgccgctgaacttcaagaaattcgccgctaaagtgtccatcgacacctcttctctgcagtatgagaacgatgacctgatgcgtccggacttcaacaacgatgactacgctattgcttgctgcgtaagcccgatgatcgttggtaaacaaatgcagttcttcggtgcgcgtgcaaacctggcgaaaaccatgctgtacgcaatcaacggcggcgttgacgaaaaactgaaaatgcaggttggtccgaagtctgaaccgatcaaaggcgatgtcctgaactatgatgaagtgatggagcgcatggatcacttcatggactggctggctaaacagtacatcactgcactgaacatcatccactacatgcacgacaagtacagctacgaagcctctctgatggcgctgcacgaccgtgacgttatccgcaccatggcgtgtggtatcgctggtctgtccgttgctgctgactccctgtctgcaatcaaatatgcgaaagttaaaccgattcgtgacgaagacggtctggctatcgacttcgaaatcgaaggcgaatacccgcagtttggtaacaatgatccgcgtgtagatgacctggctgttgacctggtagaacgtttcatgaagaaaattcagaaactgcacacctaccgtgacgctatcccgactcagtctgttctgaccatcacttctaacgttgtgtatggtaagaaaacgggtaacaccccagacggtcgtcgtgctggcgcgccgttcggaccgggtgctaacccgatgcacggtcgtgaccagaaaggtgcagtagcctctctgacttccgttgctaaactgccgtttgcttacgctaaagatggtatctcctacaccttctctatcgttccgaacgcactgggtaaagacgacgaagttcgtaagaccaacctggctggtctgatggatggttacttccaccacgaagcatccatcgaaggtggtcagcacctgaacgttaacgtgatgaaccgtgaaatgctgctcgacgcgatggaaaacccggaaaaatatccgcagctgaccatccgtgtatctggctacgcagtacgtttcaactcgctgactaaagaacagcagcaggacgttattactcgtaccttcactcaatctatgtaa

SEQ ID NO: 26 - protein, 760 aa, Escherichia coli
MSELNEKLATAWEGFTKGDWQNEVNVRDFIQKNYTPYEGDESFLAGATEATTTLWDKVMEGVKLENRTHAPVDFDTAVASTITSHDAGYINKQLEKIVGLQTEAPLKRALIPFGGIKMIEGSCKAYNRELDPMIKKIFTEYRKTHNQGVFDVYTPDILRCRKSGVLTGLPDAYGRGRIIGDYRRVALYGIDYLMKDKLAQFTSLQADLENGVNLEQTIRLREEIAEQHRALGQMKEMAAKYGYDISGPATNAQEAIQWTYFGYLAAVKSQNGAAMSFGRTSTFLDVYIERDLKAGKITEQEAQEMVDHLVMKLRMVRFLRTPEYDELFSGDPIWATESIGGMGLDGRTLVTKNSFRFLNTLYTMGPSPEPNMTILWSEKLPLNFKKFAAKVSIDTSSLQYENDDLMRPDFNNDDYAIACCVSPMIVGKQMQFFGARANLAKTMLYAINGGVDEKLKMQVGPKSEPIKGDVLNYDEVMERMDHFMDWLAKQYITALNIIHYMHDKYSYEASLMALHDRDVIRTMACGIAGLSVAADSLSAIKYAKVKPIRDEDGLAIDFEIEGEYPQFGNNDPRVDDLAVDLVERFMKKIQKLHTYRDAIPTQSVLTITSNVVYGKKTGNTPDGRRAGAPFGPGANPMHGRDQKGAVASLTSVAKLPFAYAKDGISYTFSIVPNALGKDDEVRKTNLAGLMDGYFHHEASIEGGQHLNVNVMNREMLLDAMENPEKYPQLTIRVSGYAVRFNSLTKEQQQDVITRTFTQSM

SEQ ID NO: 27 - DNA, 1203 bp, Escherichia coli
  source 1..1203: mol_type = genomic DNA
atgtcgagtaagttagtactggttctgaactgcggtagttcttcactgaaatttgccatcatcgatgcagtaaatggtgaagagtacctttctggtttagccgaatgtttccacctgcccgaagcacgtatcaaatggaaaatggacggcaataaacaggaagcggctttaggtgcaggcgccgctcacagcgaagcgctcaactttatcgttaatactattctggcacaaaaaccagaactgtctgcgcagctgactgctatcggtcaccgtatcgtacacggcggcgaaaagtataccagctccgtagtgatcgatgagtctgttattcagggtatcaaagatgcagcttcttttgcaccgctgcacaacccggctcacctgatcggtatcgaagaagctctgaaatctttcccacagctgaaagacaaaaacgttgctgtatttgacaccgcgttccaccagactatgccggaagagtcttacctctacgccctgccttacaacctgtacaaagagcacggcatccgtcgttacggcgcgcacggcaccagccacttctatgtaacccaggaagcggcaaaaatgctgaacaaaccggtagaagaactgaacatcatcacctgccacctgggcaacggtggttccgtttctgctatccgcaacggtaaatgcgttgacacctctatgggcctgaccccgctggaaggtctggtcatgggtacccgttctggtgatatcgatccggcgatcatcttccacctgcacgacaccctgggcatgagcgttgacgcaatcaacaaactgctgaccaaagagtctggcctgctgggtctgaccgaagtgaccagcgactgccgctatgttgaagacaactacgcgacgaaagaagacgcgaagcgcgcaatggacgtttactgccaccgcctggcgaaatacatcggtgcctacactgcgctgatggatggtcgtctggacgctgttgtattcactggtggtatcggtgaaaatgccgcaatggttcgtgaactgtctctgggcaaactgggcgtgctgggctttgaagttgatcatgaacgcaacctggctgcacgtttcggcaaatctggtttcatcaacaaagaaggtacccgtcctgcggtggttatcccaaccaacgaagaactggttatcgcgcaagacgcgagccgcctgactgcctga

SEQ ID NO: 28 - protein, 400 aa, Escherichia coli
MSSKLVLVLNCGSSSLKFAIIDAVNGEEYLSGLAECFHLPEARIKWKMDGNKQEAALGAGAAHSEALNFIVNTILAQKPELSAQLTAIGHRIVHGGEKYTSSVVIDESVIQGIKDAASFAPLHNPAHLIGIEEALKSFPQLKDKNVAVFDTAFHQTMPEESYLYALPYNLYKEHGIRRYGAHGTSHFYVTQEAAKMLNKPVEELNIITCHLGNGGSVSAIRNGKCVDTSMGLTPLEGLVMGTRSGDIDPAIIFHLHDTLGMSVDAINKLLTKESGLLGLTEVTSDCRYVEDNYATKEDAKRAMDVYCHRLAKYIGAYTALMDGRLDAVVFTGGIGENAAMVRELSLGKLGVLGFEVDHERNLAARFGKSGFINKEGTRPAVVIPTNEELVIAQDASRLTA

SEQ ID NO: 29 - DNA, 2145 bp, Escherichia coli
  source 1..2145: mol_type = genomic DNA
gtgtcccgtattattatgctgatccctaccggaaccagcgtcggtctgaccagcgtcagccttggcgtgatccgtgcaatggaacgcaaaggcgttcgtctgagcgttttcaaacctatcgctcagccgcgtaccggtggcgatgcgcccgatcagactacgactatcgtgcgtgcgaactcttccaccacgacggccgctgaaccgctgaaaatgagctacgttgaaggtctgctttccagcaatcagaaagatgtgctgatggaagagatcgtcgcaaactaccacgctaacaccaaagacgctgaagtcgttctggttgaaggtctggtcccgacacgtaagcaccagtttgcccagtctctgaactacgaaatcgctaaaacgctgaatgcggaaatcgtcttcgttatgtctcagggcactgacaccccggaacagctgaaagagcgtatcgaactgacccgcaacagcttcggcggtgccaaaaacaccaacatcaccggcgttatcgttaacaaactgaacgcaccggttgatgaacagggtcgtactcgcccggatctgtccgagattttcgacgactcttccaaagctaaagtaaacaatgttgatccggcgaagctgcaagaatccagcccgctgccggttctcggcgctgtgccgtggagctttgacctgatcgcgactcgtgcgatcgatatggctcgccacctgaatgcgaccatcatcaacgaaggcgacatcaatactcgccgcgttaaatccgtcactttctgcgcacgcagcattccgcacatgctggagcacttccgtgccggttctctgctggtgacttccgcagaccgtcctgacgtgctggtggccgcttgcctggcagccatgaacggcgtagaaatcggtgccctgctgctgactggcggttacgaaatggacgcgcgcatttctaaactgtgcgaacgtgctttcgctaccggcctgccggtatttatggtgaacaccaacacctggcagacctctctgagcctgcagagcttcaacctggaagttccggttgacgatcacgaacgtatcgagaaagttcaggaatacgttgctaactacatcaacgctgactggatcgaatctctgactgccacttctgagcgcagccgtcgtctgtctccgcctgcgttccgttatcagctgactgaacttgcgcgcaaagcgggcaaacgtatcgtactgccggaaggtgacgaaccgcgtaccgttaaagcagccgctatctgtgctgaacgtggtatcgcaacttgcgtactgctgggtaatccggcagagatcaaccgtgttgcagcgtctcagggtgtagaactgggtgcagggattgaaatcgttgatccagaagtggttcgcgaaagctatgttggtcgtctggtcgaactgcgtaagaacaaaggcatgaccgaaaccgttgcccgcgaacagctggaagacaacgtggtgctcggtacgctgatgctggaacaggatgaagttgatggtctggtttccggtgctgttcacactaccgcaaacaccatccgtccgccgctgcagctgatcaaaactgcaccgggcagctccctggtatcttccgtgttcttcatgctgctgccggaacaggtttacgtttacggtgactgtgcgatcaacccggatccgaccgctgaacagctggcagaaatcgcgattcagtccgctgattccgctgcggccttcggtatcgaaccgcgcgttgctatgctctcctactccaccggtacttctggtgcaggtagcgacgtagaaaaagttcgcgaagcaactcgtctggcgcaggaaaaacgtcctgacctgatgatcgacggtccgctgcagtacgacgctgcggtaatggctgacgttgcgaaatccaaagcgccgaactctccggttgcaggtcgcgctaccgtgttcatcttcccggatctgaacaccggtaacaccacctacaaagcggtacagcgttctgccgacctgatctccatcgggccgatgctgcagggtatgcgcaagccggttaacgacctgtcccgtggcgcactggttgacgatatcgtctacaccatcgcgctgactgcgattcagtctgcacagcagcagtaa

SEQ ID NO: 30 - protein, 714 aa, Escherichia coli
VSRIIMLIPTGTSVGLTSVSLGVIRAMERKGVRLSVFKPIAQPRTGGDAPDQTTTIVRANSSTTTAAEPLKMSYVEGLLSSNQKDVLMEEIVANYHANTKDAEVVLVEGLVPTRKHQFAQSLNYEIAKTLNAEIVFVMSQGTDTPEQLKERIELTRNSFGGAKNTNITGVIVNKLNAPVDEQGRTRPDLSEIFDDSSKAKVNNVDPAKLQESSPLPVLGAVPWSFDLIATRAIDMARHLNATIINEGDINTRRVKSVTFCARSIPHMLEHFRAGSLLVTSADRPDVLVAACLAAMNGVEIGALLLTGGYEMDARISKLCERAFATGLPVFMVNTNTWQTSLSLQSFNLEVPVDDHERIEKVQEYVANYINADWIESLTATSERSRRLSPPAFRYQLTELARKAGKRIVLPEGDEPRTVKAAAICAERGIATCVLLGNPAEINRVAASQGVELGAGIEIVDPEVVRESYVGRLVELRKNKGMTETVAREQLEDNVVLGTLMLEQDEVDGLVSGAVHTTANTIRPPLQLIKTAPGSSLVSSVFFMLLPEQVYVYGDCAINPDPTAEQLAEIAIQSADSAAAFGIEPRVAMLSYSTGTSGAGSDVEKVREATRLAQEKRPDLMIDGPLQYDAAVMADVAKSKAPNSPVAGRATVFIFPDLNTGNTTYKAVQRSADLISIGPMLQGMRKPVNDLSRGALVDDIVYTIALTAIQSAQQQ

SEQ ID NO: 31 - DNA, 1320 bp, Escherichia coli
  source 1..1320: mol_type = genomic DNA
atgacccatcaattaagatcgcgcgatatcatcgctctgggctttatgacatttgcgttgttcgtcggcgcaggtaacattattttccctccaatggtcggcttacaggcaggcgaacacgtctggactgcggcattcggcttcctcattactgccgttggcctgccggtattaacggtagtggcgctggcaaaagttggcggcggtgttgacagcctcagcacgccaatcggtaaagtcgctggcgtactgctggcaacggtttgttacctggcggtggggccgcttttcgctacgccgcgtacagctaccgtttcctttgaagtggggattgcgccgctgacgggtgattccgcgctgccgctgtttatctacagcctggtctatttcgctatcgttattctggtttcgctctatccgggcaagctgctggataccgtgggcaacttccttgcgccgctgaaaattatcgcgctggtcatcctgtctgttgccgctattgtctggccggcgggttctatcagcacggcgactgaggcttatcaaaacgctgcgttttctaacggcttcgttaacggctatctgaccatggatacgctgggcgcaatggtgtttggtatcgttattgttaacgcggcgcgttctcgtggcgttaccgaagcgcgtctgctgacccgttataccgtctgggctggcctgatggcgggtgttggtctgactctgctgtacctggcgctgttccgtctggggtcagacagcgcgtcgctggtcgatcagtctgcaaacggcgctgctattctgcatgcttacgttcagcacacctttggcggcggcggtagcttcctgctggcggcgttaatcttcatcgcctgcctggtaacggcagttggcctgacctgtgcttgtgcagaattctttgcccagtacgtaccgctctcttatcgtacgctggtgtttatcctcggcggcttctcgatggtggtttctaacctcggcttaagccagctgatccagatctccgtaccggtgctgaccgctatttatccgccgtgtatcgcactggttgtattaagttttacacgctcatggtggcataattcgtcccgcgtgattgctccgccgatgtttatcagcctgctttttggtattctcgacgggatcaaagcatctgcattcagcgatatcttaccgtcctgggcgcagcgtttaccgctggccgaacaaggtctggcgtggttaatgccaacagtggtgatggtggttctggccattatctgggatcgcgcggcaggtcgtcaggtgacctccagcgctcactaa

SEQ ID NO: 32 - protein, 439 aa, Escherichia coli
MTHQLRSRDIIALGFMTFALFVGAGNIIFPPMVGLQAGEHVWTAAFGFLITAVGLPVLTVVALAKVGGGVDSLSTPIGKVAGVLLATVCYLAVGPLFATPRTATVSFEVGIAPLTGDSALPLFIYSLVYFAIVILVSLYPGKLLDTVGNFLAPLKIIALVILSVAAIVWPAGSISTATEAYQNAAFSNGFVNGYLTMDTLGAMVFGIVIVNAARSRGVTEARLLTRYTVWAGLMAGVGLTLLYLALFRLGSDSASLVDQSANGAAILHAYVQHTFGGGGSFLLAALIFIACLVTAVGLTCACAEFFAQYVPLSYRTLVFILGGFSMVVSNLGLSQLIQISVPVLTAIYPPCIALVVLSFTRSWWHNSSRVIAPPMFISLLFGILDGIKASAFSDILPSWAQRLPLAEQGLAWLMPTVVMVVLAIIWDRAAGRQVTSSAH

SEQ ID NO: 33 - DNA, 1110 bp, Escherichia coli
  source 1..1110: mol_type = genomic DNA
atgaaacggaatgcgaaaactatcatcgcagggatgattgcactggcaatttcacacaccgctatggctgacgatattaaagtcgccgttgtcggcgcgatgtccggcccgattgcccagtggggcgatatggaatttaacggcgcgcgtcaggcaattaaagacattaatgccaaagggggaattaagggcgataaactggttggcgtggaatatgacgacgcatgcgacccgaaacaagccgttgcggtcgccaacaaaatcgttaatgacggcattaaatacgttattggtcatctgtgttcttcttctacccagcctgcgtcagatatctatgaagacgaaggtattctgatgatctcgccgggagcgaccaacccggagctgacccaacgcggttatcaacacattatgcgtactgccgggctggactcttcccaggggccaacggcggcaaaatacattcttgagacggtgaagccccagcgcatcgccatcattcacgacaaacaacagtatggcgaagggctggcgcgttcggtgcaggacgggctgaaagcggctaacgccaacgtcgtcttcttcgacggtattaccgccggggagaaagatttctccgcgctgatcgcccgcctgaaaaaagaaaacatcgacttcgtttactacggcggttactacccggaaatggggcagatgctgcgccaggcccgttccgttggcctgaaaacccagtttatggggccggaaggtgtgggtaatgcgtcgttgtcgaacattgccggtgatgccgccgaaggcatgttggtcactatgccaaaacgctatgaccaggatccggcaaaccagggcatcgttgatgcgctgaaagcagacaagaaagatccgtccgggccttatgtctggatcacctacgcggcggtgcaatctctggcgactgcccttgagcgtaccggcagcgatgagccgctggcgctggtgaaagatttaaaagctaacggtgcaaacaccgtgattgggccgctgaactgggatgaaaaaggcgatcttaagggatttgattttggtgtcttccagtggcacgccgacggttcatccacggcagccaagtga

SEQ ID NO: 34 - protein, 369 aa, Escherichia coli
MKRNAKTIIAGMIALAISHTAMADDIKVAVVGAMSGPIAQWGDMEFNGARQAIKDINAKGGIKGDKLVGVEYDDACDPKQAVAVANKIVNDGIKYVIGHLCSSSTQPASDIYEDEGILMISPGATNPELTQRGYQHIMRTAGLDSSQGPTAAKYILETVKPQRIAIIHDKQQYGEGLARSVQDGLKAANANVVFFDGITAGEKDFSALIARLKKENIDFVYYGGYYPEMGQMLRQARSVGLKTQFMGPEGVGNASLSNIAGDAAEGMLVTMPKRYDQDPANQGIVDALKADKKDPSGPYVWITYAAVQSLATALERTGSDEPLALVKDLKANGANTVIGPLNWDEKGDLKGFDFGVFQWHADGSSTAAK

SEQ ID NO: 35 - DNA, 927 bp, Escherichia coli
  source 1..927: mol_type = genomic DNA
atgtctgagcagtttttgtatttcttgcagcagatgtttaacggcgtcacgctgggcagtacctacgcgctgatagccatcggctacaccatggtttacggcattatcggcatgatcaacttcgcccacggcgaggtttatatgattggcagctacgtctcatttatgatcatcgccgcgctgatgatgatgggcattgataccggctggctgctggtagctgcgggattcgtcggcgcaatcgtcattgccagcgcctacggctggagtatcgaacgggtggcttaccgcccggtgcgtaactctaagcgcctgattgcactcatctctgcaatcggtatgtccatcttcctgcaaaactacgtcagcctgaccgaaggttcgcgcgacgtggcgctgccgagcctgtttaacggtcagtgggtggtggggcatagcgaaaacttctctgcctctattaccaccatgcaggcggtgatctggattgttaccttcctcgccatgctggcgctgacgattttcattcgctattcccgcatgggtcgcgcgtgtcgtgcctgcgcggaagatctgaaaatggcgagtctgcttggcattaacaccgaccgggtgattgcgctgacctttgtgattggcgcggcgatggcggcggtggcgggtgtgctgctcggtcagttctacggcgtcattaacccctacatcggctttatggccgggatgaaagcctttaccgcggcggtgctcggtgggattggcagcattccgggagcgatgattggcggcctgattctggggattgcggaggcgctctcttctgcctatctgagtacggaatataaagatgtggtctcattcgccctgctgattctggtgctgctggtgatgccgaccggtattctgggtcgcccggaggtagagaaagtatga

SEQ ID NO: 36 - protein, 308 aa, Escherichia coli
MSEQFLYFLQQMFNGVTLGSTYALIAIGYTMVYGIIGMINFAHGEVYMIGSYVSFMIIAALMMMGIDTGWLLVAAGFVGAIVIASAYGWSIERVAYRPVRNSKRLIALISAIGMSIFLQNYVSLTEGSRDVALPSLFNGQWVVGHSENFSASITTMQAVIWIVTFLAMLALTIFIRYSRMGRACRACAEDLKMASLLGINTDRVIALTFVIGAAMAAVAGVLLGQFYGVINPYIGFMAGMKAFTAAVLGGIGSIPGAMIGGLILGIAEALSSAYLSTEYKDVVSFALLILVLLVMPTGILGRPEVEKV

SEQ ID NO: 37 - DNA, 1278 bp, Escherichia coli
  source 1..1278: mol_type = genomic DNA
atgaaaccgatgcatattgcaatggcgctgctctctgccgcgatgttctttgtgctggcgggcgtctttatgggcgtgcaactggagctggatggcaccaaactggtggtcgacacggcttcggatgtccgttggcagtgggtgtttatcggcacggcggtggtctttttcttccagcttttgcgaccggctttccagaaagggttgaaaagcgtttccggaccgaagtttattctgcccgccattgatggctccacggtgaagcagaaactgttcctcgtggcgctgttggtgcttgcggtggcgtggccgtttatggtttcacgcgggacggtggatattgccaccctgaccatgatctacattatcctcggtctcgggctgaacgtggttgttggtctttctggtctgctggtgctggggtacggcggtttttacgccatcggcgcttacacttttgcgctgctcaatcactattacggcttgggcttctggacctgcctgccgattgctggattaatggcagcggcggcgggcttcctgctcggttttccggtgctgcgtttgcgcggtgactatctggcgatcgttaccctcggtttcggcgaaattgtgcgcatattgctgctcaataacaccgaaattaccggcggcccgaacggaatcagtcagatcccgaaaccgacactcttcggactcgagttcagccgtaccgctcgtgaaggcggctgggacacgttcagtaatttctttggcctgaaatacgatccctccgatcgtgtcatcttcctctacctggtggcgttgctgctggtggtgctaagcctgtttgtcattaaccgcctgctgcggatgccgctggggcgtgcgtgggaagcgttgcgtgaagatgaaatcgcctgccgttcgctgggcttaagcccgcgtcgtatcaagctgactgcctttaccataagtgccgcgtttgccggttttgccggaacgctgtttgcggcgcgtcagggctttgtcagcccggaatccttcacctttgccgaatcggcgtttgtgctggcgatagtggtgctcggcggtatgggctcgcaatttgcggtgattctggcggcaattttgctggtggtgtcgcgcgagttgatgcgtgatttcaacgaatacagcatgttaatgctcggtggtttgatggtgctgatgatgatctggcgtccgcagggcttgctgcccatgacgcgcccgcaactgaagctgaaaaacggcgcagcgaaaggagagcaggcatga

SEQ ID NO: 38 - protein, 425 aa, Escherichia coli
MKPMHIAMALLSAAMFFVLAGVFMGVQLELDGTKLVVDTASDVRWQWVFIGTAVVFFFQLLRPAFQKGLKSVSGPKFILPAIDGSTVKQKLFLVALLVLAVAWPFMVSRGTVDIATLTMIYIILGLGLNVVVGLSGLLVLGYGGFYAIGAYTFALLNHYYGLGFWTCLPIAGLMAAAAGFLLGFPVLRLRGDYLAIVTLGFGEIVRILLLNNTEITGGPNGISQIPKPTLFGLEFSRTAREGGWDTFSNFFGLKYDPSDRVIFLYLVALLLVVLSLFVINRLLRMPLGRAWEALREDEIACRSLGLSPRRIKLTAFTISAAFAGFAGTLFAARQGFVSPESFTFAESAFVLAIVVLGGMGSQFAVILAAILLVVSRELMRDFNEYSMLMLGGLMVLMMIWRPQGLLPMTRPQLKLKNGAAKGEQA

SEQ ID NO: 39 - DNA, 768 bp, Escherichia coli
  source 1..768: mol_type = genomic DNA
atgagtcagccattattatctgttaacggcctgatgatgcgcttcggcggcctgctggcggtgaacaacgtcaatcttgaactgtacccgcaggagatcgtctcgttaatcggccctaacggtgccggaaaaaccacggtttttaactgtctgaccggattctacaaacccaccggcggcaccattttactgcgcgatcagcacctggaaggtttaccggggcagcaaattgcccgcatgggcgtggtgcgcaccttccagcatgtgcgtctgttccgtgaaatgacggtaattgaaaacctgctggtggcgcagcatcagcaactgaaaaccgggctgttctctggcctgttgaaaacgccatccttccgtcgcgcccagagcgaagcgctcgaccgcgccgcgacctggcttgagcgcattggtttgctggaacacgccaaccgtcaggcgagtaacctggcctatggtgaccagcgccgtcttgagattgcccgctgcatggtgacgcagccggagattttaatgctcgacgaacctgcggcaggtcttaacccgaaagagacgaaagagctggatgagctgattgccgaactgcgcaatcatcacaacaccactatcttgttgattgaacacgatatgaagctggtgatgggaatttcggaccgaatttacgtggtcaatcaggggacgccgctggcaaacggtacgccggagcagatccgtaataacccggacgtgatccgtgcctatttaggtgaggcataa

SEQ ID NO: 40 - protein, 255 aa, Escherichia coli
MSQPLLSVNGLMMRFGGLLAVNNVNLELYPQEIVSLIGPNGAGKTTVFNCLTGFYKPTGGTILLRDQHLEGLPGQQIARMGVVRTFQHVRLFREMTVIENLLVAQHQQLKTGLFSGLLKTPSFRRAQSEALDRAATWLERIGLLEHANRQASNLAYGDQRRLEIARCMVTQPEILMLDEPAAGLNPKETKELDELIAELRNHHNTTILLIEHDMKLVMGISDRIYVVNQGTPLANGTPEQIRNNPDVIRAYLGEA

SEQ ID NO: 41 - DNA, 714 bp, Escherichia coli
  source 1..714: mol_type = genomic DNA
atggaaaaagtcatgttgtcctttgacaaagtcagcgcccactacggcaaaatccaggcgctgcatgaggtgagcctgcatatcaatcagggcgagattgtcacgctgattggcgcgaacggggcggggaaaaccaccttgctcggcacgttatgcggcgatccgcgtgccaccagcgggcgaattgtgtttgatgataaagacattaccgactggcagacagcgaaaatcatgcgcgaagcggtggcgattgtcccggaagggcgtcgcgtcttctcgcggatgacggtggaagagaacctggcgatgggcggtttttttgctgaacgcgaccagttccaggagcgcataaagtgggtgtatgagctgtttccacgtctgcatgagcgccgtattcagcgggcgggcaccatgtccggcggtgaacagcagatgctggcgattggtcgtgcgctgatgagcaacccgcgtttgctactgcttgatgagccatcgctcggtcttgcgccgattatcatccagcaaattttcgacaccatcgagcagctgcgcgagcaggggatgactatctttctcgtcgagcagaacgccaaccaggcgctaaagctggcggatcgcggctacgtgctggaaaacggccatgtagtgctttccgatactggtgatgcgctgctggcgaatgaagcggtgagaagtgcgtatttaggcgggtaa

SEQ ID NO: 42 - protein, 237 aa, Escherichia coli [sequence omitted]

SEQ ID NO: 43 - DNA, 1476 bp, Escherichia coli
  source 1..1476: mol_type = genomic DNA
atggcgaactatttcaatacactgaacttacgccaacagctggcacagcttggaaaatgccgctttatgggacgtgatgaatttgcagacggggcaagttatttacagggcaagaaagttgtgatcgtgggatgcggcgcgcagggtcttaaccagggcctgaatatgcgcgacagcggcctcgacatttcatatgccctgcgcaaggaagcgattgcggaaaagcgtgctagctggcgcaaagcgacagaaaacggttttaaagtaggcacgtacgaagagttaattccgcaagccgatctggtgatcaatctgacgcccgataaacagcacagtgacgttgtgcgcacggtacaaccgcttatgaaagatggcgccgcgttgggttatagccacggcttcaatattgtggaagtgggcgaacagattcgcaaggatattaccgtggttatggtggccccgaaatgccctggaaccgaagtacgtgaagaatataaacgcggttttggcgtaccgacactgattgcggtgcatccggagaatgatccgaagggcgaaggcatggcgattgccaaggcttgggcggcagctacgggcggccatcgcgcaggtgtcctcgaatcttcttttgtggcagaggtcaaatcggatctgatgggcgagcagaccattctgtgtggcatgctgcaagccggctcactgctgtgttttgacaaactggtagaggaaggaaccgatcctgcgtacgctgaaaagctgattcagttcggctgggaaaccattacggaagcccttaaacagggcggaattaccctgatgatggaccgcctgagcaacccggcgaaattgcgcgcctatgcattaagcgaacagttgaaagaaattatggccccgctgttccagaaacatatggatgatattatttctggcgagttctcgtcgggtatgatggccgattgggcaaacgatgacaagaaattgttaacctggcgcgaagaaacgggcaagactgctttcgaaactgcgccgcagtacgaaggcaagattggagaacaggaatacttcgataaaggcgttctgatgatcgcgatggtcaaagcgggcgttgaactggcatttgaaacgatggtggatagcggaatcattgaagaaagcgcctactacgagagcttgcatgaactgccgctgattgccaacaccatcgcgcgtaaacgcctgtatgaaatgaacgtggtgatcagtgacacagcggaatacggtaattatcttttctcatatgcttgtgtgcctcttttgaagccgttcatggccgaactgcaaccaggtgacctgggcaaagccattcccgaaggcgcagtggacaacggccagttacgcgatgtgaacgaggccattcgtagccatgccatcgaacaggtgggaaagaaacttcgtggctacatgaccgatatgaagcgcatcgccgtggccggctaa

SEQ ID NO: 44 - protein, 491 aa, Escherichia coli
MANYFNTLNLRQQLAQLGKCRFMGRDEFADGASYLQGKKVVIVGCGAQGLNQGLNMRDSGLDISYALRKEAIAEKRASWRKATENGFKVGTYEELIPQADLVINLTPDKQHSDVVRTVQPLMKDGAALGYSHGFNIVEVGEQIRKDITVVMVAPKCPGTEVREEYKRGFGVPTLIAVHPENDPKGEGMAIAKAWAAATGGHRAGVLESSFVAEVKSDLMGEQTILCGMLQAGSLLCFDKLVEEGTDPAYAEKLIQFGWETITEALKQGGITLMMDRLSNPAKLRAYALSEQLKEIMAPLFQKHMDDIISGEFSSGMMADWANDDKKLLTWREETGKTAFETAPQYEGKIGEQEYFDKGVLMIAMVKAGVELAFETMVDSGIIEESAYYESLHELPLIANTIARKRLYEMNVVISDTAEYGNYLFSYACVPLLKPFMAELQPGDLGKAIPEGAVDNGQLRDVNEAIRSHAIEQVGKKLRGYMTDMKRIAVAG

SEQ ID NO: 45 - DNA, 1851 bp, Escherichia coli
  source 1..1851: mol_type = genomic DNA
atgcctaagtaccgttccgccaccaccactcatggtcgtaatatggcgggtgctcgtgcgctgtggcgcgccaccggaatgaccgacgccgatttcggtaagccgattatcgcggttgtgaactcgttcacccaatttgtaccgggtcacgtccatctgcgcgatctcggtaaactggtcgccgaacaaattgaagcggctggcggcgttgccaaagagttcaacaccattgcggtggatgatgggattgccatgggccacggggggatgctttattcactgccatctcgcgaactgatcgctgattccgttgagtatatggtcaacgcccactgcgccgacgccatggtctgcatctctaactgcgacaaaatcaccccggggatgctgatggcttccctgcgcctgaatattccggtgatctttgtttccggcggcccgatggaggccgggaaaaccaaactttccgatcagatcatcaagctcgatctggttgatgcgatgatccagggcgcagacccgaaagtatctgactcccagagcgatcaggttgaacgttccgcgtgtccgacctgcggttcctgctccgggatgtttaccgctaactcaatgaactgcctgaccgaagcgctgggcctgtcgcagccgggcaacggctcgctgctggcaacccacgccgaccgtaagcagctgttccttaatgctggtaaacgcattgttgaattgaccaaacgttattacgagcaaaacgacgaaagtgcactgccgcgtaatatcgccagtaaggcggcgtttgaaaacgccatgacgctggatatcgcgatgggtggatcgactaacaccgtacttcacctgctggcggcggcgcaggaagcggaaatcgacttcaccatgagtgatatcgataagctttcccgcaaggttccacagctgtgtaaagttgcgccgagcacccagaaataccatatggaagatgttcaccgtgctggtggtgttatcggtattctcggcgaactggatcgcgcggggttactgaaccgtgatgtgaaaaacgtacttggcctgacgttgccgcaaacgctggaacaatacgacgttatgctgacccaggatgacgcggtaaaaaatatgttccgcgcaggtcctgcaggcattcgtaccacacaggcattctcgcaagattgccgttgggatacgctggacgacgatcgcgccaatggctgtatccgctcgctggaacacgcctacagcaaagacggcggcctggcggtgctctacggtaactttgcggaaaacggctgcatcgtgaaaacggcaggcgtcgatgacagcatcctcaaattcaccggcccggcgaaagtgtacgaaagccaggacgatgcggtagaagcgattctcggcggtaaagttgtcgccggagatgtggtagtaattcgctatgaaggcccgaaaggcggtccggggatgcaggaaatgctctacccaaccagcttcctgaaatcaatgggtctcggcaaagcctgtgcgctgatcaccgacggtcgtttctctggtggcacctctggtctttccatcggccacgtctcaccggaagcggcaagcggcggcagcattggcctgattgaagatggtgacctgatcgctatcgacatcccgaaccgtggcattcagttacaggtaagcgatgccgaactggcggcgcgtcgtgaagcgcaggacgctcgaggtgacaaagcctggacgccgaaaaatcgtgaacgtcaggtctcctttgccctgcgtgcttatgccagcctggcaaccagcgccgacaaaggcgcggtgcgcgataaatcgaaactggggggttaa

SEQ ID NO: 46 - protein, 616 aa, Escherichia coli
MPKYRSATTTHGRNMAGARALWRATGMTDADFGKPIIAVVNSFTQFVPGHVHLRDLGKLVAEQIEAAGGVAKEFNTIAVDDGIAMGHGGMLYSLPSRELIADSVEYMVNAHCADAMVCISNCDKITPGMLMASLRLNIPVIFVSGGPMEAGKTKLSDQIIKLDLVDAMIQGADPKVSDSQSDQVERSACPTCGSCSGMFTANSMNCLTEALGLSQPGNGSLLATHADRKQLFLNAGKRIVELTKRYYEQNDESALPRNIASKAAFENAMTLDIAMGGSTNTVLHLLAAAQEAEIDFTMSDIDKLSRKVPQLCKVAPSTQKYHMEDVHRAGGVIGILGELDRAGLLNRDVKNVLGLTLPQTLEQYDVMLTQDDAVKNMFRAGPAGIRTTQAFSQDCRWDTLDDDRANGCIRSLEHAYSKDGGLAVLYGNFAENGCIVKTAGVDDSILKFTGPAKVYESQDDAVEAILGGKVVAGDVVVIRYEGPKGGPGMQEMLYPTSFLKSMGLGKACALITDGRFSGGTSGLSIGHVSPEAASGGSIGLIEDGDLIAIDIPNRGIQLQVSDAELAARREAQDARGDKAWTPKNRERQVSFALRAYASLATSADKGAVRDKSKLGG

SEQ ID NO: 47 - DNA, 1689 bp, Escherichia coli
  source 1..1689: mol_type = genomic DNA
atggcaagttcgggcacaacatcgacgcgtaagcgctttaccggcgcagaatttatcgttcatttcctggaacagcagggcattaagattgtgacaggcattccgggcggttctatcctgcctgtttacgatgccttaagccaaagcacgcaaatccgccatattctggcccgtcatgaacagggcgcgggctttatcgctcagggaatggcgcgcaccgacggtaaaccggcggtctgtatggcctgtagcggaccgggtgcgactaacctggtgaccgccattgccgatgcgcggctggactccatcccgctgatttgcatcactggtcaggttcccgcctcgatgatcggcaccgacgccttccaggaagtggacacctacggcatctctatccccatcaccaaacacaactatctggtcagacatatcgaagaactcccgcaggtcatgagcgatgccttccgcattgcgcaatcaggccgcccaggcccggtgtggatagacattcctaaggatgtgcaaacggcagtttttgagattgaaacacagcccgctatggcagaaaaagccgccgcccccgcctttagcgaagaaagcattcgtgacgcagcggcgatgattaacgctgccaaacgcccggtgctttatctgggcggcggtgtgatcaatgcgcccgcacgggtgcgtgaactggcggagaaagcgcaactgcctaccaccatgactttaatggcgctgggcatgttgccaaaagcgcatccgttgtcgctgggtatgctggggatgcacggcgtgcgcagcaccaactatattttgcaggaggcggatttgttgatagtgctcggtgcgcgttttgatgaccgggcgattggcaaaaccgagcagttctgtccgaatgccaaaatcattcatgtcgatatcgaccgtgcagagctgggtaaaatcaagcagccgcacgtggcgattcaggcggatgttgatgacgtgctggcgcagttgatcccgctggtggaagcgcaaccgcgtgcagagtggcaccagttggtagcggatttgcagcgtgagtttccgtgtccaatcccgaaagcgtgcgatccgttaagccattacggcctgatcaacgccgttgccgcctgtgtcgatgacaatgcaattatcaccaccgacgttggtcagcatcagatgtggaccgcgcaagcttatccgctcaatcgcccacgccagtggctgacctccggtgggctgggcacgatgggttttggcctgcctgcggcgattggcgctgcgctggcgaacccggatcgcaaagtgttgtgtttctccggcgacggcagcctgatgatgaatattcaggagatggcgaccgccagtgaaaatcagctggatgtcaaaatcattctgatgaacaacgaagcgctggggctggtgcatcagcaacagagtctgttctacgagcaaggcgtttttgccgccacctatccgggcaaaatcaactttatgcagattgccgccggattcggcctcgaaacctgtgatttgaataacgaagccgatccgcaggcttcattgcaggaaatcatcaatcgccctggcccggcgctgatccatgtgcgcattgatgccgaagaaaaagtttacccgatggtgccgccaggtgcggcgaatactgaaatggtgggggaataa

SEQ ID NO: 48 - protein, 562 aa, Escherichia coli
MASSGTTSTRKRFTGAEFIVHFLEQQGIKIVTGIPGGSILPVYDALSQSTQIRHILARHEQGAGFIAQGMARTDGKPAVCMACSGPGATNLVTAIADARLDSIPLICITGQVPASMIGTDAFQEVDTYGISIPITKHNYLVRHIEELPQVMSDAFRIAQSGRPGPVWIDIPKDVQTAVFEIETQPAMAEKAAAPAFSEESIRDAAAMINAAKRPVLYLGGGVINAPARVRELAEKAQLPTTMTLMALGMLPKAHPLSLGMLGMHGVRSTNYILQEADLLIVLGARFDDRAIGKTEQFCPNAKIIHVDIDRAELGKIKQPHVAIQADVDDVLAQLIPLVEAQPRAEWHQLVADLQREFPCPIPKACDPLSHYGLINAVAACVDDNAIITTDVGQHQMWTAQAYPLNRPRQWLTSGGLGTMGFGLPAAIGAALANPDRKVLCFSGDGSLMMNIQEMATASENQLDVKIILMNNEALGLVHQQQSLFYEQGVFAATYPGKINFMQIAAGFGLETCDLNNEADPQASLQEIINRPGPALIHVRIDAEEKVYPMVPPGAANTEMVGE

SEQ ID NO: 49 - DNA, 291 bp, Escherichia coli
  source 1..291: mol_type = genomic DNA
atgcaaaacacaactcatgacaacgtaattctggagctcaccgttcgcaaccatccgggcgtaatgacccacgtttgtggcctttttgcccgccgcgcttttaacgttgaaggcattctttgtctgccgattcaggacagcgacaaaagccatatctggctactggtcaatgacgaccagcgtctggagcagatgataagccaaatcgataagctggaagatgtcgtgaaagtgcagcgtaatcagtccgatccgacgatgtttaacaagatcgcggtgttttttcagtaa

SEQ ID NO: 50 - protein, 96 aa, Escherichia coli
MQNTTHDNVILELTVRNHPGVMTHVCGLFARRAFNVEGILCLPIQDSDKSHIWLLVNDDQRLEQMISQIDKLEDVVKVQRNQSDPTMFNKIAVFFQ

SEQ ID NO: 51 - DNA, 291 bp, Escherichia coli
  source 1..291: mol_type = other DNA
atgcaaaacacaactcatgacaacgtaattctggagctcaccgttcgcaaccatccggatgacttcacccacgtttgtggcctttttgcccgccgcgcttttaacgttgaaggcattctttgtctgccgattcaggacagcgacaaaagccatatctggctactggtcaatgacgaccagcgtctggagcagatgataagccaaatcgataagctggaagatgtcgtgaaagtgcagcgtaatcagtccgatccgacgatgtttaacaagatcgcggtgttttttcagtaa

SEQ ID NO: 52 - protein, 96 aa, Escherichia coli
MQNTTHDNVILELTVRNHPDDFTHVCGLFARRAFNVEGILCLPIQDSDKSHIWLLVNDDQRLEQMISQIDKLEDVVKVQRNQSDPTMFNKIAVFFQ

SEQ ID NO: 53 - DNA, 1158 bp, Streptomyces aureofaciens
  source 1..1158: mol_type = genomic DNA
gtgaccgacgtacagaccccggccacgcaccccgcacccggcgtgctcagcaggatcttccgcaccgagcaggacggcgcccccggcgacggccacgagcaggtcgtcctctgccacgaccgcagctccgggctcaaggcgatcatcgccatccactccaccgccctcggccccgccctcggcggcacccgcttcttcccgtacgcctccgaggaggcggccctggaggacgcgctgaacctctcccgcgggatgtcctacaagaacgcgctcgccggcctcgacctcggcggcggcaaggccgtcatcatcggtgacccgaacaaggacaagaacgaggcgatgctccgggcctacggccgcttcgtcgagtccctgcgcggccgctacatcaccgcctgcgacgtgggcacctacgtccaggacatggacgtcatcgcgcgcgagaccgagttcgtgaccggccgctcgcccgagcacggcggcgccggcgactcgtcgatcctgaccgccttcggcgtcttccagggcatgcgcgcctccgcccaggcccgttggggccagcccaccctgcgcggcaagcgggtcggcgtcgcgggcgtcggcaaggtcggccactacctggtcggccacctggtcgcggacggcgccaccgtggtcgtcaccgaccccttcgaggccgccgtcaaccgcgtgcgcgcggcccacccggaggtggaggtggtcgccgacaccaccgccctgctccaggccaagctggacgtctacgccccctgcgccctcggcggcgcgctgaccgaccccgtggtcgcggcgctcagcgagttcggcacctccatcgtctgcggcgcggccaacaaccagctggcccacccgggcgtcgagaaggacctgtccgaccgcggcatcctctacgcccccgactacctggtgaactccggcggcgtgatccaggtcgccgacgagatcgacggcttcaacttcgagcgggccaagaacaaggccaccaagatcttcgacaccaccctggagatcttcacccgggcagccgccgacggcgtcccgccggcggtcgccgccgaccgcctggccgagaagcggatgcgcgagatcagcgcgctgcgcacggtcctgctgccgggcgcccgccgcggctga

SEQ ID NO: 54 - protein, 385 aa, Streptomyces aureofaciens
MTDVQTPATHPAPGVLSRIFRTEQDGAPGDGHEQVVLCHDRSSGLKAIIAIHSTALGPALGGTRFFPYASEEAALEDALNLSRGMSYKNALAGLDLGGGKAVIIGDPNKDKNEAMLRAYGRFVESLRGRYITACDVGTYVQDMDVIARETEFVTGRSPEHGGAGDSSILTAFGVFQGMRASAQARWGQPTLRGKRVGVAGVGKVGHYLVGHLVADGATVVVTDPFEAAVNRVRAAHPEVEVVADTTALLQAKLDVYAPCALGGALTDPVVAALSEFGTSIVCGAANNQLAHPGVEKDLSDRGILYAPDYLVNSGGVIQVADEIDGFNFERAKNKATKIFDTTLEIFTRAAADGVPPAVAADRLAEKRMREISALRTVLLPGARRG

SEQ ID NO: 55 - DNA, 1158 bp, Streptomyces aureofaciens
  source 1..1158: mol_type = other DNA
atgaccgacgttcagaccccggcgacccacccggcgccgggcgtgctgagccgtatctttcgtaccgagcaggatggtgcgccgggtgatggtcacgaacaagtggttctgtgccacgatcgtagcagcggcctgaaggcgatcattgcgatccacagcaccgcgctgggtccggcgctgggtggcacccgtttctttccgtatgcgagcgaggaagcggcgctggaggatgcgctgaacctgagccgtggcatgagctataagaacgcgctggcgggtctggatctgggtggcggtaaagcggttatcattggtgacccgaacaaggataaaaacgaggcgatgctgcgtgcgtacggccgtttcgtggaaagcctgcgtggtcgttacatcaccgcgtgcgacgttggtacctatgtgcaggacatggatgttattgcgcgtgagaccgaatttgtgaccggccgtagcccggagcatggcggtgcgggtgatagcagcatcctgaccgcgttcggcgtttttcagggtatgcgtgcgagcgcgcaggcgcgttggggtcaaccgaccctgcgtggcaagcgtgttggtgtggcgggcgttggtaaagtgggccactacctggttggtcatctggtggcggatggtgcgaccgtggttgtgaccgatccgttcgaagcggcggttaaccgtgtgcgtgcggcgcacccggaagttgaggtggtggcggacaccaccgcgctgctgcaagcgaagctggatgtgtatgcgccgtgcgcgctgggcggtgcgctgaccgacccggttgtggcggcgctgagcgagtttggcaccagcatcgtttgcggtgcggcgaacaaccagctggcgcacccgggcgtggaaaaagacctgagcgatcgtggtattctgtacgcgccggactatctggttaacagcggcggtgttatccaagtggcggacgagattgatggcttcaactttgaacgtgcgaagaacaaagcgaccaagatcttcgataccaccctggaaatttttacccgtgcggcggcggatggtgttccgccggcggtggcggcggatcgtctggcggagaaacgtatgcgtgaaattagcgcgctgcgtaccgtgctgctgccgggtgcgcgtcgtggttaa

SEQ ID NO: 56 - DNA, 930 bp, Escherichia coli
  source 1..930: mol_type = genomic DNA
atgaccacgaagaaagctgattacatttggttcaatggggagatggttcgctgggaagacgcgaaggtgcatgtgatgtcgcacgcgctgcactatggcacttcggtttttgaaggcatccgttgctacgactcgcacaaaggaccggttgtattccgccatcgtgagcatatgcagcgtctgcatgactccgccaaaatctatcgcttcccggtttcgcagagcattgatgagctgatggaagcttgtcgtgacgtgatccgcaaaaacaatctcaccagcgcctatatccgtccgctgatcttcgtcggtgatgttggcatgggagtaaacccgccagcgggatactcaaccgacgtgattatcgctgctttcccgtggggagcgtatctgggcgcagaagcgctggagcaggggatcgatgcgatggtttcctcctggaaccgcgcagcaccaaacaccatcccgacggcggcaaaagccggtggtaactacctctcttccctgctggtgggtagcgaagcgcgccgccacggttatcaggaaggtatcgcgctggatgtgaacggttatatctctgaaggcgcaggcgaaaacctgtttgaagtgaaagatggtgtgctgttcaccccaccgttcacctcctccgcgctgccgggtattacccgtgatgccatcatcaaactggcgaaagagctgggaattgaagtacgtgagcaggtgctgtcgcgcgaatccctgtacctggcggatgaagtgtttatgtccggtacggcggcagaaatcacgccagtgcgcagcgtagacggtattcaggttggcgaaggccgttgtggcccggttaccaaacgcattcagcaagccttcttcggcctcttcactggcgaaaccgaagataaatggggctggttagatcaagttaatcaataa

SEQ ID NO: 57 - protein, 309 aa, Escherichia coli
MTTKKADYIWFNGEMVRWEDAKVHVMSHALHYGTSVFEGIRCYDSHKGPVVFRHREHMQRLHDSAKIYRFPVSQSIDELMEACRDVIRKNNLTSAYIRPLIFVGDVGMGVNPPAGYSTDVIIAAFPWGAYLGAEALEQGIDAMVSSWNRAAPNTIPTAAKAGGNYLSSLLVGSEARRHGYQEGIALDVNGYISEGAGENLFEVKDGVLFTPPFTSSALPGITRDAIIKLAKELGIEVREQVLSRESLYLADEVFMSGTAAEITPVRSVDGIQVGEGRCGPVTKRIQQAFFGLFTGETEDKWGWLDQVNQ

SEQ ID NO: 58 - DNA, 738 bp, Escherichia coli
  source 1..738: mol_type = genomic DNA
atggaaagccctactccacagcctgctcctggttcggcgaccttcatggaaggatgcaaagacagtttaccgattgttattagttatattccggtggcctttgcgttcggtctgaatgcgacccgtctgggattctctcctctcgaaagcgtttttttctcctgcatcatttatgcaggcgcgagccagttcgtcattaccgcgatgctggcagccgggagtagtttgtggattgctgcactgaccgtcatggcaatggatgttcgccatgtgttgtatggcccgtcactgcgtagccgtattattcagcgtctgcaaaaatcgaaaaccgccctgtgggcgtttggcctgacggatgaggtttttgccgccgcaaccgcaaaactggtacgcaataatcgccgctggagcgagaactggatgatcggcattgccttcagttcatggtcatcgtgggtatttggtacggtaataggggcattctccggcagcggcttgctgcaaggttatcccgccgttgaagctgcattaggttttatgcttccggcactctttatgagtttcctgctcgcctctttccagcgcaaacaatctctttgcgttaccgcagcgttagttggtgcccttgcaggcgtaacgctattttctattcccgtcgccattctggcaggcattgtctgtggctgcctcactgcgttaatccaggcattctggcaaggagcgcccgatgagctatga

SEQ ID NO: 59 - protein, 245 aa, Escherichia coli
MESPTPQPAPGSATFMEGCKDSLPIVISYIPVAFAFGLNATRLGFSPLESVFFSCIIYAGASQFVITAMLAAGSSLWIAALTVMAMDVRHVLYGPSLRSRIIQRLQKSKTALWAFGLTDEVFAAATAKLVRNNRRWSENWMIGIAFSSWSSWVFGTVIGAFSGSGLLQGYPAVEAALGFMLPALFMSFLLASFQRKQSLCVTAALVGALAGVTLFSIPVAILAGIVCGCLTALIQAFWQGAPDEL

SEQ ID NO: 60 - DNA, 336 bp, Escherichia coli
  source 1..336: mol_type = genomic DNA
atgagctatgaggttctgctgcttgggttactagttggcgtggcgaattattgcttccgctatttgccgctgcgcctgcgtgtgggtaatgcccgcccaaccaaacgtggcgcggtaggtattttgctcgacaccattggcatcgcctcgatatgcgctctgctggttgtctctaccgcaccagaagtgatgcacgatacacgccgtttcgtgcccacgctggtcggcttcgcggtactgggtgccagtttctataaaacacgcagcattatcatcccaacactgcttagtgcgctggcctatgggctcgcctggaaagtgatggcgattatataa

SEQ ID NO: 61 - protein, 111 aa, Escherichia coli
MSYEVLLLGLLVGVANYCFRYLPLRLRVGNARPTKRGAVGILLDTIGIASICALLVVSTAPEVMHDTRRFVPTLVGFAVLGASFYKTRSIIIPTLLSALAYGLAWKVMAII

SEQ ID NO: 62 - DNA, 1248 bp, Escherichia coli
  source 1..1248: mol_type = genomic DNA
atggcactgaatattccattcagaaatgcgtactatcgttttgcatccagttactcatttctcttttttatttcctggtcgctgtggtggtcgttatacgctatttggctgaaaggacatctaggattaacagggacggaattaggtacactttattcggtcaaccagtttaccagcattctatttatgatgttctacggcatcgttcaggataaactcggtctgaagaaaccgctcatctggtgtatgagtttcattctggtcttgaccggaccgtttatgatttacgtttatgaaccgttactgcaaagcaatttttctgtaggtctaattctgggggcgctcttttttggcctggggtatctggcgggatgcggtttgcttgacagcttcaccgaaaaaatggcgcgaaattttcatttcgaatatggaacagcgcgcgcctggggatcttttggctatgctattggcgcgttctttgccggtatattttttagtatcagtccccatatcaacttctggttggtctcgctatttggcgctgtatttatgatgatcaacatgcgttttaaagataaggatcaccagtgcatagcggcggatgcgggaggggtaaaaaaagaggattttatcgcagttttcaaggatcgaaacttctgggttttcgtcatatttattgtggggacgtggtctttctataacatttttgatcaacaactctttcctgtcttttatgcaggtttattcgaatcacacgatgtaggaacgcgcctgtatggttatctcaactcattccaggtggtactcgaagcgctgtgcatggcgattattcctttctttgtgaatcgggtagggccaaaaaatgcattacttatcggtgttgtgattatggcgttgcgtatcctttcctgcgcgttgttcgttaacccctggattatttcattagtgaagctgttacatgccattgaggttccactttgtgtcatatccgtcttcaaatacagcgtggcaaactttgataagcgcctgtcgtcgacgatctttctgattggttttcaaattgccagttcgcttgggattgtgctgctttcaacgccgactgggatactctttgaccacgcaggctaccagacagttttcttcgcaatttcgggtattgtctgcctgatgttgctatttggcattttcttcctgagtaaaaaacgcgagcaaatagttatggaaacgcctgtaccttcagcaatatag

SEQ ID NO: 63 - protein, 415 aa, Escherichia coli
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCIAADAGGVKKEDFIAVFKDRNFWVFVIFIVGTWSFYNIFDQQLFPVFYAGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 64 - DNA, 1434 bp, Escherichia coli
  source 1..1434: mol_type = genomic DNA
atgacgcaatctcgattgcatgcggcgcaaaacgcactagcaaaacttcacgagcgccgaggtaacactttctatccccattttcacctcgcgcctcctgccgggtggatgaacgatccaaacggcctgatctggtttaacgatcgttatcacgcgttttatcaacatcacccgatgagcgaacactgggggccaatgcactggggacatgccaccagcgacgatatgatccactggcagcatgagcctattgcgctagcgccaggagacgagaatgacaaagacgggtgtttttcaggtagtgctgtcgatgacaatggtgtcctctcacttatctacaccggacacgtctggctcgatggtgcaggtaatgacgatgcaattcgcgaagtacaatgtctggctaccagtcgggatggtattcatttcgagaaacagggtgtgatcctcactccaccagaaggcatcatgcacttccgcgatcctaaagtgtggcgtgaagccgacacatggtggatggtagtcggggcgaaagacccaggcaacacggggcagatcctgctttatcgcggcagttcattgcgtgaatggactttcgatcgcgtactggcccacgctgatgcgggtgaaagctatatgtgggaatgtccggactttttcagccttggcgatcagcattatctgatgttttccccgcagggaatgaatgccgagggatacagttatcgaaatcgctttcaaagtggcgtaatacccggaatgtggtcgccaggacgactttttgcacaatccgggcattttactgaacttgataacgggcatgacttttatgcaccacaaagctttgtagcgaaggatggtcggcgtattgttatcggctggatggatatgtgggaatcgccaatgccctcaaaacgtgaaggctgggcaggctgcatgacgctggcgcgcgagctatcagagagcaatggcaaactcctacaacgcccggtacacgaagctgagtcgttacgccagcagcatcaatctatctctccccgcacaatcagcaataaatatgttttgcaggaaaacgcgcaagcagttgagattcagttgcagtgggcgctgaagaacagtgatgccgaacattacggattacagctcggcgctggaatgcggctgtatattgataaccaatctgagcgacttgttttgtggcggtattacccacacgagaatttagatggctaccgtagtattcccctcccgcagggtgacatgctcgccctaaggatatttatcgatacatcatccgtggaagtatttattaacgacggggaggcggtgatgagtagccgaatatatccgcagccagaagaacgggaactgtcgctctatgcctcccacggagtggctgtgctgcaacatggagcactctggcaactgggttaa

SEQ ID NO: 65 - protein, 477 aa, Escherichia coli
MTQSRLHAAQNALAKLHERRGNTFYPHFHLAPPAGWMNDPNGLIWFNDRYHAFYQHHPMSEHWGPMHWGHATSDDMIHWQHEPIALAPGDENDKDGCFSGSAVDDNGVLSLIYTGHVWLDGAGNDDAIREVQCLATSRDGIHFEKQGVILTPPEGIMHFRDPKVWREADTWWMVVGAKDPGNTGQILLYRGSSLREWTFDRVLAHADAGESYMWECPDFFSLGDQHYLMFSPQGMNAEGYSYRNRFQSGVIPGMWSPGRLFAQSGHFTELDNGHDFYAPQSFVAKDGRRIVIGWMDMWESPMPSKREGWAGCMTLARELSESNGKLLQRPVHEAESLRQQHQSISPRTISNKYVLQENAQAVEIQLQWALKNSDAEHYGLQLGAGMRLYIDNQSERLVLWRYYPHENLDGYRSIPLPQGDMLALRIFIDTSSVEVFINDGEAVMSSRIYPQPEERELSLYASHGVAVLQHGALWQLG

SEQ ID NO: 66 - DNA, 924 bp, Escherichia coli
  source 1..924: mol_type = genomic DNA
atgtcagccaaagtatgggttttaggggatgcggtcgtagatctcttgccagaatcagacgggcggctactgccttgtcctggcggcgcgccagctaacgttgcggtgggaatcgccagattaggcggaacaagtgggtttataggtcgggtcggtgatgatccttttggtgcgttaatgcaaagaacgctgctaactgagggtgtcgatatcacgtatctgaagcaagatgaatggcaccggacatccacggtgcttgtcgatctgaacgatcaaggagaacgttcatttacgtttatggtccgccccagtgccgatctttttttagagacgacagacttgccctgctggcgacatggcgaatggttacatctctgttcaattgcgttgtctgccgagccttcgcgtaccagcgcatttactgcgatgacggcgatccggcatgccggaggttttgtcagcttcgatcccaatattcgtgaagatctatggcaagacgagcatttgctccgcttgtgtttgcggcaggcgctacaactggcggatgtcgtcaagctctcggaagaagaatggcgacttatcagtggaaaaacacagaacgatcgggatatatgcgccctggcaaaagagtatgagatcgccatgctgttggtgactaaaggtgcagaaggggtggtggtctgttatcgaggacaagtccaccattttgctggaatgtctgtgaattgtgtcgatagcactggggcgggagatgcgttcgttgccgggttactcacaggtctgtcctctacgggattatctacagatgagagagaaatgcgacgaattatcgatctcgctcaacgttgcggagcgcttgcagtaacagcgaaaggggcaatgacagcgctgccatgtcgacaagaactggaaagtgagaagtaa

SEQ ID NO: 67 - protein, 307 aa, Escherichia coli
MSAKVWVLGDAVVDLLPESDGRLLPCPGGAPANVAVGIARLGGTSGFIGRVGDDPFGALMQRTLLTEGVDITYLKQDEWHRTSTVLVDLNDQGERSFTFMVRPSADLFLETTDLPCWRHGEWLHLCSIALSAEPSRTSAFTAMTAIRHAGGFVSFDPNIREDLWQDEHLLRLCLRQALQLADVVKLSEEEWRLISGKTQNDRDICALAKEYEIAMLLVTKGAEGVVVCYRGQVHHFAGMSVNCVDSTGAGDAFVAGLLTGLSSTGLSTDEREMRRIIDLAQRCGALAVTAKGAMTALPCRQELESEK

SEQ ID NO: 68 - DNA, 996 bp, Escherichia coli
  source 1..996: mol_type = genomic DNA
atggcttcattaaaggatgtcgcacgcctggcgggagtgtcgatgatgacagtctcccgggtgatgcataatgcagaatctgtgcgtcctgcaacgcgtgaccgcgtattgcaggcaatccagaccctgaattatgttcctgatctttccgcccgtaagatgcgcgctcaaggacgtaagccgtcgactctcgccgtgctggcgcaggacactgctaccactcctttctctgttgatattctgcttgccattgagcaaaccgccagcgagttcggctggaatagttttttaatcaatattttttctgaagatgacgctgcccgcgcggcacgtcagctgcttgcccaccgtccggatggcattatctatactacaatggggctgcgacatatcacgctgcctgagtctctgtatggtgaaaatattgtattggcgaactgtgtggcggatgacccagcgttacccagttatatccctgatgattacactgcacaatatgaatcaacacagcatttgctcgcggcgggctatcgtcaaccgttatgcttctggctaccggaaagtgcgttggcaacagggtatcgtcggcagggatttgagcaggcctggcgtgatgctggacgagatctggctgaggtgaaacaatttcacatggcaacaggtgatgatcactacaccgatctcgcaagtttactcaatgcccacttcaaatcgggcaaaccagattttgatgttctgatatgtggtaacgatcgcgcagcctttgtggcttatcaggttcttttggcgaagggggtacgtatcccgcaggatgtcgccgtaatgggctttgataatctggttggcgtcgggcatctgtttttaccgccgctgaccacaattcagcttccacatgacattatcgggcgggaagctgcattgcatattattgaaggtcgtgaagggggaagagtgacgcggatcccttgcccgctgttgatccgttgttccacctga

SEQ ID NO: 69 - protein, 331 aa, Escherichia coli
MASLKDVARLAGVSMMTVSRVMHNAESVRPATRDRVLQAIQTLNYVPDLSARKMRAQGRKPSTLAVLAQDTATTPFSVDILLAIEQTASEFGWNSFLINIFSEDDAARAARQLLAHRPDGIIYTTMGLRHITLPESLYGENIVLANCVADDPALPSYIPDDYTAQYESTQHLLAAGYRQPLCFWLPESALATGYRRQGFEQAWRDAGRDLAEVKQFHMATGDDHYTDLASLLNAHFKSGKPDFDVLICGNDRAAFVAYQVLLAKGVRIPQDVAVMGFDNLVGVGHLFLPPLTTIQLPHDIIGREAALHIIEGREGGRVTRIPCPLLIRCST

SEQ ID NO: 70 - DNA, 924 bp, Salmonella sp.
  source 1..924: mol_type = genomic DNA
atgaatgcaaaagtttgggttctgggcgacgcggtggtggacctgctgccggagagcgaagggcgcctgctgcagtgccctggaggcgcgccggctaacgtggcggtaggggttgcccgccttggcggcaacagcggatttatcggcgccgtcggcggtgacccgtttggccgctacatgcgtcataccctgcaacaggagcaggtcgacgtcagccatatgtatctcgacgatcagcaccgcacgtccactgtggtcgtcgaccttgacgaccagggggaacgcacctttacctttatggtacgccccagcgcggacctgttcctggttgaagaagacctgccacagtttgccgccggacagtggttgcacgtctgctccatcgcgctcagcgccgagcccagccgtagcactaccttcgcggcgatggagagcatcaggtctgccggcggtcgggtcagctttgaccctaatattcgtcccgatctctggcaggatcaggctttgctgctagcctgcctcgatcgcgctttgcacatggccaacgtggtaaagctatcggaagaggagctggtcttcatcagcagcagtaatgatttagcatacggaatcgccagcgtaacggagcgctatcagccagaattgctactggtgacccggggcaaagcgggggtgcttgccgcgtttcagcagaagtttacccatttcaacgcccggcctgtggccagcgtggacaccaccggcgcgggagacgcatttgtcgccggactgctcgccagccttgcggctaacgggatgccaacggacatgaccgcactggaaccgacactcacgcttgcacagacctgcggcgccctggccaccacagccaaaggtgcgatgaccgccttgccttatcagcgcgatctcaaccgtcagttttaa

SEQ ID NO: 71 - protein, 307 aa, Salmonella sp.
MNAKVWVLGDAVVDLLPESEGRLLQCPGGAPANVAVGVARLGGNSGFIGAVGGDPFGRYMRHTLQQEQVDVSHMYLDDQHRTSTVVVDLDDQGERTFTFMVRPSADLFLVEEDLPQFAAGQWLHVCSIALSAEPSRSTTFAAMESIRSAGGRVSFDPNIRPDLWQDQALLLACLDRALHMANVVKLSEEELVFISSSNDLAYGIASVTERYQPELLLVTRGKAGVLAAFQQKFTHFNARPVASVDTTGAGDAFVAGLLASLAANGMPTDMTALEPTLTLAQTCGALATTAKGAMTALPYQRDLNRQF

SEQ ID NO: 72 - DNA, 1518 bp, Salmonella sp.
  source 1..1518: mol_type = genomic DNA
atgtacagaaaaagcacacttgcgatgcttatcgctttgctaaccagcgctgcctcagcccatgcgcaaacggatataagcaccattgaagcccgactcaacgcgctggaaaaacgcctgcaggaggcagaaaacagggcgcaaacggcggaaaaccgcgccggggcggcggagaaaaaagttcagcaactcaccgcgcagcagcaaaaaaaccagaactcgactcaggaagtggctcagcgtaccgccagacttgagaaaaaagccgatgacaaaagcggatttgagtttcacggttacgcccgctccggcgtgataatgaatgattccggcgccagcaccaaatccggagcctacataacgccggcaggtgaaaccggcggagctatcggccgtctgggaaaccaggccgatacctatgttgaaatgaatcttgaacataagcagaccctggataatggggccacgacccgctttaaggtgatggtcgccgacgggcaaacctcttataacgactggactgcaagcaccagcgatctgaacgttcgtcaggcctttgtcgaattgggtaacctgccgacgttcgctgggccatttaagggctccaccctgtgggccgggaaacgtttcgaccgcgacaatttcgatattcactggattgactctgatgtcgtgttcctcgccggtaccggtggtggtatctatgacgtgaagtggaacgacggcctgcggagtaatttctccctgtacgggcgtaacttcggcgacattgatgattccagcaacagcgtgcagaactatatcctcaccatgaatcacttcgcaggtccgctgcagatgatggtcagcggtctgcgggcgaaggataacgacgagcgtaaagatagcaacggcaatctggcaaaaggcgatgcggcaaacaccggcgtgcatgcgctgctcggcctgcataacgacagtttctacggcctgcgcgacggtagcagtaaaaccgctctgctttatggtcatggtctgggcgcagaggttaaaggtatcggatctgatggcgcacttcgtccgggagccgacacatggcgcattgccagttacggcaccacgccgctcagcgaaaactggtctgttgccccggcaatgctggcgcaacgcagtaaagaccgctatgccgatggcgacagctatcagtgggcaacattcaacctgcgtctgattcaggcaatcaatcagaatttcgctctcgcctacgaaggcagctaccagtacatggatcttaaacccgaaggttataacgatcgtcaggcggtgaacggtagcttctacaagctcaccttcgccccgacatttaaggtcggcagtatcggtgatttcttcagtcgcccggagattcgtttctatacctcctggatggactggagcaaaaaactgaataattacgccagcgacgacgccctgggcagtgacggttttaactcgggcggcgaatggtctttcggtgtgcagatggaaacctggttctga

SEQ ID NO: 73 - protein, 505 aa, Salmonella sp.
MYRKSTLAMLIALLTSAASAHAQTDISTIEARLNALEKRLQEAENRAQTAENRAGAAEKKVQQLTAQQQKNQNSTQEVAQRTARLEKKADDKSGFEFHGYARSGVIMNDSGASTKSGAYITPAGETGGAIGRLGNQADTYVEMNLEHKQTLDNGATTRFKVMVADGQTSYNDWTASTSDLNVRQAFVELGNLPTFAGPFKGSTLWAGKRFDRDNFDIHWIDSDVVFLAGTGGGIYDVKWNDGLRSNFSLYGRNFGDIDDSSNSVQNYILTMNHFAGPLQMMVSGLRAKDNDERKDSNGNLAKGDAANTGVHALLGLHNDSFYGLRDGSSKTALLYGHGLGAEVKGIGSDGALRPGADTWRIASYGTTPLSENWSVAPAMLAQRSKDRYADGDSYQWATFNLRLIQAINQNFALAYEGSYQYMDLKPEGYNDRQAVNGSFYKLTFAPTFKVGSIGDFFSRPEIRFYTSWMDWSKKLNNYASDDALGSDGFNSGGEWSFGVQMETWF

SEQ ID NO: 74 - DNA, 1371 bp, Salmonella sp.
  source 1..1371: mol_type = genomic DNA
atggattttgaacagatttcctgctcgctgcttccgcttcttggaggcaaagaaaatatcgccagcgccgcgcactgcgccacgcgcctgcgcctggtgctggtcgatgattcgctggccgaccagcaggccatcggcaaagttgaaggggtgaagggctgttttcgtaatgccggacagatgcagattattttcggcaccggggtggtaaataaggtctacgctgcctttactcaggcggcgggtattagcgaatccagcaaatcggaagccgccgacatcgcggcaaaaaagctcaatccgttccagcgcatcgcccgcctgctatcaaacatcttcgtgccgataatccctgccatcgtcgcctctggtctgctgatgggcctgctgggaatggtcaaaacatacggctgggttgacccgggcaacgccatctacatcatgctggatatgtgcagctcggcggcatttatcattctgccgattctgattggctttaccgccgcccgcgaattcggcggtaatccttatctcggcgcgacgcttggcggcattctgactcatccagcgctgactaacgcctggggcgtggccgcgggtttccacaccatgaactttttcggcttcgaaattgccatgatcggctatcagggtacggtgttcccggtactgctggcagtatggtttatgagcatcgttgagaagcagttgcgtcgcgcaatccccgatgccctggatttgatcctgacgccgttcctgacggtgattatatccggttttatcgccctgttgattatcggcccggccggtcgcgcactgggcgacggtatctcgtttgtcctcagcaccctgattagccacgccggctggctcgccgggttactgtttggcggtctctattcagttatcgtcattaccggtattcatcacagcttccatgcggttgaagccgggttgctgggcaatccctccatcggcgtcaacttcctgctgccgatttgggcgatggccaacgtcgctcagggcggagcctgtctggcggtgtggttcaaaaccaaagatgcaaaaattaaagccattactctgccctcggcgttttccgccatgctgggcatcaccgaggcggcgatttttggtattaacctgcgctttgtgaagccatttattgcggcgctgattggtggtgcggcgggcggcgcatgggtggtatctgtacacgtctacatgaccgcggtcggcttgacagcgatccccggcatggccatcgtgcaggccagttcgctgttgaactacattatcgggatggttatcgcctttggcgtcgcctttacggtctccctggttttgaaatacaaaacggacgctgaataa

SEQ ID NO: 75 - protein, 456 aa, Salmonella sp.
MDFEQISCSLLPLLGGKENIASAAHCATRLRLVLVDDSLADQQAIGKVEGVKGCFRNAGQMQIIFGTGVVNKVYAAFTQAAGISESSKSEAADIAAKKLNPFQRIARLLSNIFVPIIPAIVASGLLMGLLGMVKTYGWVDPGNAIYIMLDMCSSAAFIILPILIGFTAAREFGGNPYLGATLGGILTHPALTNAWGVAAGFHTMNFFGFEIAMIGYQGTVFPVLLAVWFMSIVEKQLRRAIPDALDLILTPFLTVIISGFIALLIIGPAGRALGDGISFVLSTLISHAGWLAGLLFGGLYSVIVITGIHHSFHAVEAGLLGNPSIGVNFLLPIWAMANVAQGGACLAVWFKTKDAKIKAITLPSAFSAMLGITEAAIFGINLRFVKPFIAALIGGAAGGAWVVSVHVYMTAVGLTAIPGMAIVQASSLLNYIIGMVIAFGVAFTVSLVLKYKTDAE

SEQ ID NO: 76 - DNA, 1401 bp, Salmonella sp.
  source 1..1401: mol_type = genomic DNA
atgtctcttccatcacgactgcctgcgattttgcaggccgtaatgcagggccagccgcgcgcgctggccgatagccactatccgcgctggcaccatgcgccggtcaccgggctgatgaacgaccccaacggctttatcgaatttgccggacgctatcatctgttttatcagtggaacccgctcgcctgcgatcatacgtttaagtgctgggcgcactggagttccatcgatctgctgcactggcagcatgagcccattgcgctgatgccggacgaagagtatgaccgtaacggctgctactccggcagcgcggtggataacaacggtacgcttaccctgtgctataccggcaacgtgaagtttgccgagggagggcgaaccgcctggcaatgcctggcaacggaaaacgctgacggcaccttccgcaaaatcggtccggtcctgccgctgccggagggctacaccggccacgtgcgcgacccaaaagtctggcgacacgaagacctgtggtacatggtgctgggcgcgcaggatcggcaaaagcgcggcaaggtgctgctgttcagctctgcggatctccatcagtggacgagtatgggtgaaatcgccggccacggcatcaatggcctcgacgacgtcggctatatgtgggagtgcccggatctttttccactcggcgaccagcatattctaatctgctgtccgcaggggattgcccgtgaggaagagtgctacctgaacacctacccggcagtatggatggcgggcgagtttgattacgctgctggcgctttcagacacggcgaactgcacgaactggacgccgggtttgagttctacgccccgcaaaccatgcttaccagtgatggccgtcgtctgctggtcggctggatgggcgtgccggagggcgaagagatgcttcagccgaccctgaacaacggctggatccatcagatgacctgcctgcgtgagctggagtttatcaacggtcagctctatcagcgtccgctacgggaactgagcgccctgcgcggtgaagcgaacggctggtcggggaacgccctgccgctggcaccgatggaaatcgatttgcaaacccgcgggggcgatatgttgagcctcgattttggcggcgtattaacccttgagtgcgatgccagcggactccgcctggcccgacgcagtctcgccagtgacgagatgcattatcgttactggcgcggaaacgtccgctcgctgcgtgttttcatcgaccagtcgagcgtggagattttcataaacggcggtgaaggggtgatgagcagccgctacttcccggcctgctccggtcagctaacattctccggcatcacgccggacgcattctgctactggccgctgcgaacttgcatggtagaataa

SEQ ID NO: 77 - protein, 466 aa, Salmonella sp.
MSLPSRLPAILQAVMQGQPRALADSHYPRWHHAPVTGLMNDPNGFIEFAGRYHLFYQWNPLACDHTFKCWAHWSSIDLLHWQHEPIALMPDEEYDRNGCYSGSAVDNNGTLTLCYTGNVKFAEGGRTAWQCLATENADGTFRKIGPVLPLPEGYTGHVRDPKVWRHEDLWYMVLGAQDRQKRGKVLLFSSADLHQWTSMGEIAGHGINGLDDVGYMWECPDLFPLGDQHILICCPQGIAREEECYLNTYPAVWMAGEFDYAAGAFRHGELHELDAGFEFYAPQTMLTSDGRRLLVGWMGVPEGEEMLQPTLNNGWIHQMTCLRELEFINGQLYQRPLRELSALRGEANGWSGNALPLAPMEIDLQTRGGDMLSLDFGGVLTLECDASGLRLARRSLASDEMHYRYWRGNVRSLRVFIDQSSVEIFINGGEGVMSSRYFPACSGQLTFSGITPDAFCYWPLRTCMVE

SEQ ID NO: 78 - DNA, 1005 bp, Salmonella sp.
  source 1..1005: mol_type = genomic DNA
atgaaaaccaaacgcgtaaccattaaagatatagccgaacaggctggcgtctccaaagcgaccgccagcttggtactgaatggtcgtggcaaggagctgcgcgtggcgcaggaaacgcgtgagcgcgtactgtcgattgcccgtaagcatcactatcagccaagcattcatgcccgctcgctgcgcaacaaccgcagccacaccatcgggctggtggtgccggagatcaccaaccacggctttgcggtctttgcccatgagctggagatgctgtgccgcgaggcgggcgtccagctgttgatctcttgtactgatgaaaaccccggtcaggagagcgtggtggtcaataatatgattgcccgccaggtcgacgggatgatcgtcgcttcctgtatgcacaacgatgccgactatctcaaactcagccaacagctgccagtggtgctgtttgaccggtgccccaatgaaagcgcgctgccgctggtaatgaccgattcgattaccccaacggcggaactgatttcccgcatcgcgcctcagcatagcgatgagttctggtttttaggcggtcaggcgcgtctgtcgccctcccgcgatcgtctgaccgggttcacgcagggtttggctcaggcgggtattgccctgcgcccggaatgggtgatcaacggcaattaccaccccagctccggctatgagatgtttgccgcactctgcgcgcgccttgggcggccgcctaaggcgctattcaccgccgcctgcgggctgctcgaaggggttctgcgctatatgagccagcaccatttactcgattccgatattcatctgacgagctttgacgatcactatctttatgattcgctgtcgctgcgtatcgacactgtccagcaggataatcgccagctggcctggcactgctacgatctgataagccagctgatcgagggcgatacgcccgaaacgctacaacgctacctgcccgcaaccctgcagtttcggcatcagtaa

SEQ ID NO: 79 - protein, 334 aa, Salmonella sp.
MKTKRVTIKDIAEQAGVSKATASLVLNGRGKELRVAQETRERVLSIARKHHYQPSIHARSLRNNRSHTIGLVVPEITNHGFAVFAHELEMLCREAGVQLLISCTDENPGQESVVVNNMIARQVDGMIVASCMHNDADYLKLSQQLPVVLFDRCPNESALPLVMTDSITPTAELISRIAPQHSDEFWFLGGQARLSPSRDRLTGFTQGLAQAGIALRPEWVINGNYHPSSGYEMFAALCARLGRPPKALFTAACGLLEGVLRYMSQHHLLDSDIHLTSFDDHYLYDSLSLRIDTVQQDNRQLAWHCYDLISQLIEGDTPETLQRYLPATLQFRHQ

SEQ ID NO: 80 - DNA, 714 bp, phage lambda
  source 1..714: mol_type = genomic DNA
atgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttacaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctaagcttagaacctttaccaaaggtgatgcggagagatgggtaagcacaaccaaaaaagccagtgattctgcattctggcttgaggttgaaggtaattccatgaccgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctga

SEQ ID NO: 81 - protein, 237 aa, phage lambda
MSTKKKPLTQEQLEDARRLKAIYEKKKNELGLSQESVADKMGMGQSGVGALFNGINALNAYNAALLTKILKVSVEEFSPSIAREIYEMYEAVSMQPSLRSEYEYPVFSHVQAGMFSPKLRTFTKGDAERWVSTTKKASDSAFWLEVEGNSMTAPTGSKPSFPDGMLILVDPEQAVEPGDFCIARLGGDEFTFKKLIRDSGQVFLQPLNPQYPMIPCNESCSVVGKVIASQWPEETFG

SEQ ID NO: 82 - DNA, 2241 bp, Escherichia coli
  source 1..2241: mol_type = genomic DNA
atgaacaagaagattcattccctggccttgttggtcaatctggggatttatggggtagcgcaggcacaagagccgaccgatactcctgtttcacatgacgatactattgtcgttaccgccgccgagcagaacttacaggcgcctggcgtttcgaccatcaccgcagatgaaatccgcaaaaacccggttgcccgcgatgtgtcgaagatcatccgtaccatgccaggcgttaacctgaccggtaactccaccagtggtcagcgtgggaataaccgacagattgatattcgcggtatgggtccggaaaacacgctgattttgattgacggcaagccggtaagcagccgtaactcggtgcgtcagggctggcgtggcgagcgcgatacccgtggtgatacttcctgggtgccacctgaaatgattgaacgtattgaagttctgcgtggtccggcagctgcgcgttatggcaacggcgcggcgggcggcgtggttaacatcattaccaaaaaaggcagcggcgagtggcacggctcctgggacgcatatttcaatgcgccagaacataaagaggaaggtgccaccaaacgcactaactttagcctgaccggtccgctgggcgacgaattcagcttccgtttgtatggcaacctcgacaaaacccaggctgacgcgtgggatatcaaccagggccatcagtccgcgcgtgccggaacgtatgccacgacgttaccagccgggcgcgaaggggtaatcaacaaagatattaatggcgtggtgcgctgggatttcgcgccattgcaatcgctggaactggaagcaggttacagccgccagggtaacctgtatgcgggcgacacccagaataccaactccgattcctatacccgctcgaaatatggcgatgaaaccaaccgtctgtatcgccagaactacgcgctgacctggaacggtggctgggataacggcgtgaccaccagcaactgggtgcagtacgaacacacccgtaactcgcgtattccggaaggtctggcgggcggtaccgaagggaaatttaacgaaaaagcgacacaggatttcgtcgatatcgatcttgatgacgtgatgctgcacagcgaagttaacctgccgattgatttcctcgttaaccagacgctgacgctgggtacggagtggaatcagcaacggatgaaggacttaagttccaacacccaggcactgaccggaacgaataccggtggcgctattgatggcgtgagtaccaccgaccgtagcccgtattcaaaagcagaaattttctcgctgtttgccgaaaacaacatggagctgactgacagcaccatcgtaacgccggggctgcgtttcgatcatcacagtattgtcggcaataactggagcccggcgctgaacatatcgcaaggtttaggcgatgacttcacgctgaaaatgggcatcgcccgtgcttataaagcgccgagcctgtaccagactaacccgaactacattctctacagtaaaggtcagggttgctatgccagcgcgggcggctgctatctgcaaggtaacgatgacctgaaagcagaaaccagcatcaacaaagagattggtctggagttcaaacgcgacgggtggctggcgggcgtcacctggttccgtaacgattatcgcaataagattgaagcgggctatgtggctgtagggcaaaacgcagtcggcaccgatctctatcagtgggataacgtgccgaaagcggtggttgaaggtctggaaggatcgttaaacgtaccggttagcgaaacggtgatgtggaccaataacatcacttatatgctgaagagtgaaaacaaaaccacgggcgaccgtttgtcgatcatcccggagtatacgttgaactcaacgctgagctggcaggcacgggaagatttgtcgatgcaaacgaccttcacctggtacggcaagcagcagccgaagaagtacaactataaaggtcagccagcggttggaccggaaaccaaagaaattagtccttacagcattgttggcctgagcgcgacctgggatgtgacgaagaatgtcagtctgaccggcggcgtggacaatctgttcgacaaacgtttgtggcgtgcgggtaatgcccagaccacgggcgatttggcaggggccaactatatcgccggtgccggggcgtatacctataacgagccgggacgtacgtggtatatgagcgtaaacacccacttctga

SEQ ID NO: 83 - protein, 746 aa, Escherichia coli
MNKKIHSLALLVNLGIYGVAQAQEPTDTPVSHDDTIVVTAAEQNLQAPGVSTITADEIRKNPVARDVSKIIRTMPGVNLTGNSTSGQRGNNRQIDIRGMGPENTLILIDGKPVSSRNSVRQGWRGERDTRGDTSWVPPEMIERIEVLRGPAAARYGNGAAGGVVNIITKKGSGEWHGSWDAYFNAPEHKEEGATKRTNFSLTGPLGDEFSFRLYGNLDKTQADAWDINQGHQSARAGTYATTLPAGREGVINKDINGVVRWDFAPLQSLELEAGYSRQGNLYAGDTQNTNSDSYTRSKYGDETNRLYRQNYALTWNGGWDNGVTTSNWVQYEHTRNSRIPEGLAGGTEGKFNEKATQDFVDIDLDDVMLHSEVNLPIDFLVNQTLTLGTEWNQQRMKDLSSNTQALTGTNTGGAIDGVSTTDRSPYSKAEIFSLFAENNMELTDSTIVTPGLRFDHHSIVGNNWSPALNISQGLGDDFTLKMGIARAYKAPSLYQTNPNYILYSKGQGCYASAGGCYLQGNDDLKAETSINKEIGLEFKRDGWLAGVTWFRNDYRNKIEAGYVAVGQNAVGTDLYQWDNVPKAVVEGLEGSLNVPVSETVMWTNNITYMLKSENKTTGDRLSIIPEYTLNSTLSWQAREDLSMQTTFTWYGKQQPKKYNYKGQPAVGPETKEISPYSIVGLSATWDVTKNVSLTGGVDNLFDKRLWRAGNAQTTGDLAGANYIAGAGAYTYNEPGRTWYMSVNTHF